(19) United States
(12) Patent Application Publication
(10) Pub. No.: US 2009/0024042 A1
(43) Pub. Date: Jan. 22, 2009

(54) METHOD AND SYSTEM FOR MONITORING VENTRICULAR FUNCTION OF A HEART

(75) Inventors: Anthony I. Nunez, Beachwood, OH (US); Harry D. Rowland, East Peoria, IL (US)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: ENDOTRONIX, INC., Peoria, IL (US)

(21) Appl. No.: 12/167,001

(22) Filed: Jul. 2, 2008

Related U.S. Application Data
(60) Provisional application No. 60/947,925, filed on Jul. 3, 2007.

(51) Int. Cl. A61B 5/0215

(52) U.S. Cl. 600/486

(57) ABSTRACT

A method for monitoring a right atrial pressure (RAP) and a left atrial pressure (LAP) for diagnosis of a heart condition includes positioning a transseptal device with respect to a pulmonary artery to monitor at least one flow characteristic of blood through the pulmonary artery. The transseptal device is configured to generate one or more signals representative of the at least one flow characteristic. A right ventricular end diastolic pressure (RVEDP) and a left ventricular end diastolic pressure (LVEDP) are detected to facilitate monitoring the heart condition.

### CROSS REFERENCE TO RELATED APPLICATIONS

This application claims the benefit of U.S. Provisional Application No. 60/947,925, filed Jul. 3, 2007, which is hereby incorporated by reference in its entirety.

### BACKGROUND OF THE INVENTION

The subject matter disclosed herein generally relates to a method and a system to facilitate measuring and responding to various parameters of a patient's health, such as the central venous pressure of the patient, and, more particularly, to a method and a system for measuring and responding to a change in the internal fluid pressure within one or more chambers of the patient's heart.

The medical industry has long been using blood pressure data to diagnose ill patients. Conventional methods generally included the use of a rubber cuff that is inflatable to restrict the flow of blood momentarily through a patient's artery. The cuff is slowly deflated to allow the blood to pass through the artery thereby creating an audible noise that is heard by the physician with a stethoscope. The pressure at which the sound is no longer heard is subsequently correlated to the pressure seen in the patient's cardiovascular system. Although this method is quick and painless, it may result in inaccurate readings as well as an inability to determine time varying cardiac chamber pressures.

Other conventional devices may be used to measure various pressures within the human or animal body including arterial pressure, venous pressure, pulmonary artery pressure, bladder pressure, left ventricle pressure or the intracranial pressure. However, such devices have limited data transmission abilities. For example, the pressure may be initially detected through the use of a pressure transmission catheter filled with a pressure transmitting medium. The pressure signal created in the pressure transmitting medium is then communicated to a transducer and subsequently a connecting catheter, which carries the signal to a signal processing and telemetry circuit. Although this system may work in design, additional system components are placed within the body to provide a conditioned signal of value to the practicing physician.

### BRIEF DESCRIPTION OF THE INVENTION

In one aspect, a method is provided for monitoring a right atrial pressure (RAP) and a left atrial pressure (LAP) for diagnosis of a heart condition. The method includes positioning a transseptal device with respect to a pulmonary artery to monitor at least one flow characteristic of blood through the pulmonary artery. The transseptal device is configured to generate one or more signals representative of the at least one flow characteristic. A right ventricular end diastolic pressure (RVEDP) and a left ventricular end diastolic pressure (LVEDP) are detected to facilitate monitoring the heart condition.

In another aspect, a system for diagnosing a heart condition is provided. The system includes a transseptal device positioned with respect to a pulmonary artery to monitor at least one flow characteristic of blood through the pulmonary artery. The transseptal device is configured to generate at least one signal representative of the at least one flow characteristic to facilitate monitoring a right atrial pressure (RAP) and a left atrial pressure (LAP). An external reader is operatively coupled to the transseptal device and configured to: receive the at least one generated signal from the transseptal device representative of the at least one flow characteristic; process the at least one received signals; and generate an appropriate output representative of the at least one flow characteristic of blood through the pulmonary artery.

### BRIEF DESCRIPTION OF THE DRAWINGS

FIG. 1 is a perspective view of an exemplary implantable sensing unit;

FIG. 2 is a perspective view of an alternative exemplary implantable sensing unit;

FIG. 3 is an exploded perspective view of an exemplary implantable medical device including sensor arrays;

FIG. 4 is a perspective view of an exemplary sensor array for use with the implantable medical device shown in FIG. 3 in a closed or collapsed configuration;

FIG. 5 is a perspective view of an exemplary sensor array for use with the implantable medical device shown in FIG. 3 in a deployed configuration;

FIG. 6 is a side view of the implantable medical device shown in FIG. 3 in an insertion configuration;

FIG. 7 is a side view of the implantable medical device shown in FIG. 3 in a deployed configuration;

FIG. 8 is a perspective view of an alternative exemplary sensor array for use with the implantable medical device shown in FIG. 3 in a deployed configuration;

FIG. 9 is a perspective view of the sensor array shown in FIG. 8 in a partially closed or collapsed configuration;

FIG. 10 is a perspective view of the sensor array shown in FIG. 8 in a fully closed or collapsed configuration;

FIG. 11 is a perspective view of an alternative exemplary implantable medical device including sensor arrays in a deployed configuration;

FIG. 12 is a perspective view of the implantable medical device shown in FIG. 11 in a closed or collapsed configuration;

FIG. 13 is a perspective view of an alternative exemplary implantable medical device including a sensing unit;

FIG. 14 is a side view of the implantable medical device shown in FIG. 13;

FIG. 15 is a front view of the implantable medical device shown in FIG. 13;

FIG. 16 is a partial cross-sectional side view of the implantable medical device shown in FIG. 15 along sectional line A-A;

FIG. 17 is an exploded perspective view of the implantable medical device shown in FIG. 13;

FIG. 18 shows a Right Atrial Pressure (RAP) waveform signal;

FIGS. 19A and 19B show a flow diagram describing steps of a method to facilitate a diagnosis of a heart condition;

FIG. 20 shows a blood pressure waveform described in a sinusoidal pattern;

FIGS. 21 and 22 show a change in vectors between each point that governs an overall shape of the curve shown in FIG. 20; and

FIGS. 23 and 24 illustrate a summation of forces on a final vector (direction) of a blood pressure curve related to an eventual angle of the vector.

### DETAILED DESCRIPTION OF THE INVENTION

The embodiments described herein provide an implantable medical device, such as a transseptal device, and method to facilitate measuring an internal fluid pressure within a chamber of a patient's heart, such as within at least one of the right ventricle, the left ventricle, the right atrium and the left atrium of a patient's heart. In one embodiment, the pressure sensor is incorporated into other areas of a patient's central venous system to measure the central venous pressure of the patient. In a particular embodiment, the device utilizes an inductor and capacitor circuitry in a parallel configuration emitting a radio frequency when charged. The emitted radio frequency is proportional to a pressure detected at a surface of the sensor. The sensor facilitates measuring various parameters of a patient's health through its output.

The implantable medical device, such as a transseptal device, includes one or more sensing units, such as a sensor array, each having one or more sensors that sense or measure physical, chemical and/or physiological parameters or variables within the respective heart chamber to facilitate obtaining data for cardiac blood pressure analysis, temperature analysis, blood chemical analysis, blood osmolar analysis, and cellular count analysis, for example. The sensing unit is configured to transmit the measurement data wirelessly to an external receiver. In one embodiment, the data is transmitted wirelessly to an external hand-held transceiver unit or reader for patient monitoring. The external reader incorporates an RF filter allowing multiple sensors to be read independently. In an alternative embodiment, the data is transmitted wirelessly to an intermediate RF link prior to being transmitted to an external unit. The intermediate RF link may be a flexible electronic or flexible printed electronic telemetry patch placed on the patient's skin or subcutaneously.

Each sensor may be a pressure sensor, an optical sensor, a biochemical sensor, a protein sensor, a motion sensor (e.g., an accelerometer or a gyroscope), a temperature sensor, a chemical sensor (e.g., a pH sensor), a biochemical sensor, and/or a genetic sensor, for example. In one embodiment, the implantable medical device includes one or more pressure sensors that are fabricated using a suitable microelectromechanical systems (MEMS) technology that utilizes a resonating frequency of an LC resonator. In alternative embodiments, the implantable medical device includes one or more sensors that function as capacitive, inductive, piezoelectric or piezoresistive sensors. The sensors may include hermetically sealed chambers that provide stable, accurate, long term monitoring. In further embodiments, the implantable medical device includes measurement electronics, such as on an application specific integrated circuit (ASIC), for sensing and/or processing the signal of the sensor. In one embodiment, the ASIC includes a sensor. In an alternative embodiment, the ASIC is separate from the sensor.

Although the following disclosure describes a cardiac pressure sensing unit including one or more sensors that measure and/or monitor a blood pressure within at least one heart chamber of the patient to facilitate obtaining data for cardiac blood pressure analysis, it should be apparent to those skilled in the art and guided by the teachings herein provided that the sensing unit and/or the sensors as described herein may be suitable for use with an implantable medical device to measure one or more physical, chemical, and/or physiological parameters or variables to facilitate obtaining data for pressure analysis, temperature analysis, blood chemical analysis, blood osmolar analysis, and cellular count analysis, for example. In an alternative embodiment, the sensing unit is implanted into one or more alternative areas of the patient's central venous system to measure the central venous pressure of the patient. In a particular embodiment, the sensor utilizes an inductor and capacitor circuitry in a parallel configuration emitting a radio frequency when charged. The emitted radio frequency is proportional to the pressure being placed on a surface of the sensor. The sensor facilitates measuring various parameters of a patient's health through its output.

MEMS sensors provide a pressure sensing device with the ability to transmit data wirelessly in a compact package. An exemplary MEMS pressure sensor is an LC tank circuit, wherein the sensor includes an inductor (L) and a capacitor (C) connected together in parallel. The sensor will resonate at a specific resonant frequency when in the presence of electromagnetic fields. The geometry of the sensor allows for the deformation of one of the capacitive plates with increased pressure. This deformation leads to a deflection of the plate, changing the capacitance value of the system and hence changing the resonant frequency of the LC circuit. This resonating frequency may be picked up by an external wireless receiver and deciphered into a correlative pressure reading.

In certain embodiment, the MEMS sensors have wireless data transmission ability. Further, the MEMS sensors are powered through electromagnetic (EM) fields directed towards the inductor coil. These EM fields charge the circuit to its maximum capacitance level depending on its environmental pressure. When the EM field is removed, the stored charge in the capacitor charges the inductor coil. This oscillating circuit produces Radio Frequency (RF) signals, which are proportional to the capacitance of the pressure sensor. The inductor coil serves as an inductor creating the oscillating RF signals having a frequency proportional to the capacitance of the pressure sensor at a certain pressure. The inductor coil also serves as an antenna coil emitting the RF signal generated by the LC tank circuitry. The system may be calibrated using a referencing sensor in the system that accommodates for a drift that occurs due to a deposition of thrombus and/or an endothelization process. Alternatively, the system may be calibrated against atmospheric pressure.

Through the use of sensors, such as one or more pressure sensors, the practicing physician can obtain valuable data regarding the physical, chemical, and/or physiological status of the patient. Obtained data is advantageously utilized for the formulation of blood pressure, heart rate, rhythm analysis, volume wave forms, central venous pressure, right ventricle systolic pressure, DP/DT, eDP/DT, as well as cardiac output index. The formulation of these readings enables the physician to diagnose and/or treat the patient.

These devices are generally implanted with the use of a catheter deployment system. Such deployment systems are commonly used in the medical industry for treatment of such diseases as abdominal aortic aneurysm (AAA) and thoracic aortic aneurysm (TAA), as well as for the implantation of pacemakers and internal defibrillators. For AAA and TAA, the treatment usually includes inserting a stent into the disease stricken portion of the aorta. The stent is then expanded inside the aorta to counteract disease-induced localized flow constrictions. Once the stent is in place, the catheter deployment system is removed from the body. This system greatly reduces the risk seen by the patient relative to traditional techniques of conventional surgery to repair the disease stricken lumen.

In order to counteract significant neointimal formation, biocompatible materials such as inactivated unfractionated heparin and/or drug eluting wells with slow release polymers combined with Tacrolimus, Sirolimus or other suitable antimetabolites may be used. Through the use of drug eluting stent technology, the probability of the lumen experiencing significant neointimal formation is greatly reduced. Drug eluting technology incorporates various biocompatible materials that can be coated on the structural elements of the devices to counteract the potential for neointimal to adhere to and/or grow on the structural elements.

The biocompatible materials may also facilitate occlusion of perforations seen in the atrium septum, such as Patent Foramen Ovale (PFO). The existence of such congenital defect is usually developed pre-birth and is seen as an abnormality instead of a disease. PFO is quite common; however few cases ultimately develop major complications such as strokes.

Various techniques may be used to determine the presence of a PFO including electrocardiograms, X-rays, echocardiograms, Doppler Ultrasound, magnetic resonance imaging (MRI), cardiac catheterization, angiography and saline bubble studies. Once a PFO has been identified, it is important to determine the size of the PFO perforation. A common method for determining the size of the PFO perforation is to incorporate a balloon study. During the balloon study, a balloon is inserted through the perforation created by the PFO and subsequently sized by a transesophageal echocardiography technique.

The deployment of the device may utilize the effects of shape memory materials, such as a nickel titanium shape memory alloy, commonly referred to by its trade name Nitinol. Shape memory alloys may work through a phase transformation from crystalline martensite to austenite. The phase transformation may result from an increase in the internal energy of the material through heat transfer. As the energy increases, the shape memory alloy has the ability to change its crystalline structure from a less organized martensite structure to a more organized austenite structure. The actual temperatures at which this phase transformation takes place can be specified through changes in the exact composition of its constituent components. In general, Nitinol alloy includes a composition of 55% Nickel and 45% Titanium allowing the final composition to exhibit the shape memory characteristics through increased heat. In addition to the shape memory and super elasticity benefits of Nitinol, the material also has inherent biocompatibility. Although the composition generally contains about 55% Nickel, a substantial amount of Nickel does not leach into the body while in contact with blood. Instead, the material exhibits biocompatibility traits commonly seen in pure titanium. This biocompatibility is due to the fact that the material can form a titanium oxide layer which can be created through electrosurface treatments. This surface treatment allows the Nickel atoms to be greatly decreased along the materials surface being replaced with Titanium Oxide layers exhibiting the better biocompatibility properties of pure Titanium.

In one embodiment, one or more pressure sensors are located within the patient's heart to allow the physician to gather valuable information regarding the physiological status of the patient's cardiovascular system. The obtained pressure waveforms allow the physician to inspect alternate areas of the patient's health including A peaks and V peaks in addition to x, x′ and y ascent and descent rates. Through analysis of these data features, the practicing physician will be able to gain valuable information regarding possible illnesses including congestive heart failure (CHF), rhythmic anomalies, beat anomalies, and cardiac output/index problems. In addition to the added benefit of diagnosing illnesses, the use of such pressure readings may also aid in future research into the formulation and chronic regression of certain illnesses as well as effects that other illnesses may have on the cardiovascular system.

The devices described herein may be fabricated using a suitable manufacturing technique including, without limitation, microfabrication of a silicon material, a fused silica material, and/or a polymeric material. During the polymeric microfabrication process, desired structures may be embossed or injection molded, or the desired structures may be formed using soft lithography. These materials may then be polymerized or cross-linked using a suitable process, such as UV lithography or through catalyst reactions.

Additionally, conductive materials may be fabricated onto the base structure through advanced techniques, such as Chemical Vapor Deposition (CVD), Physical Vapor Deposition (PVD), electrodeposition, and printing processes of metallic inks or slurries.

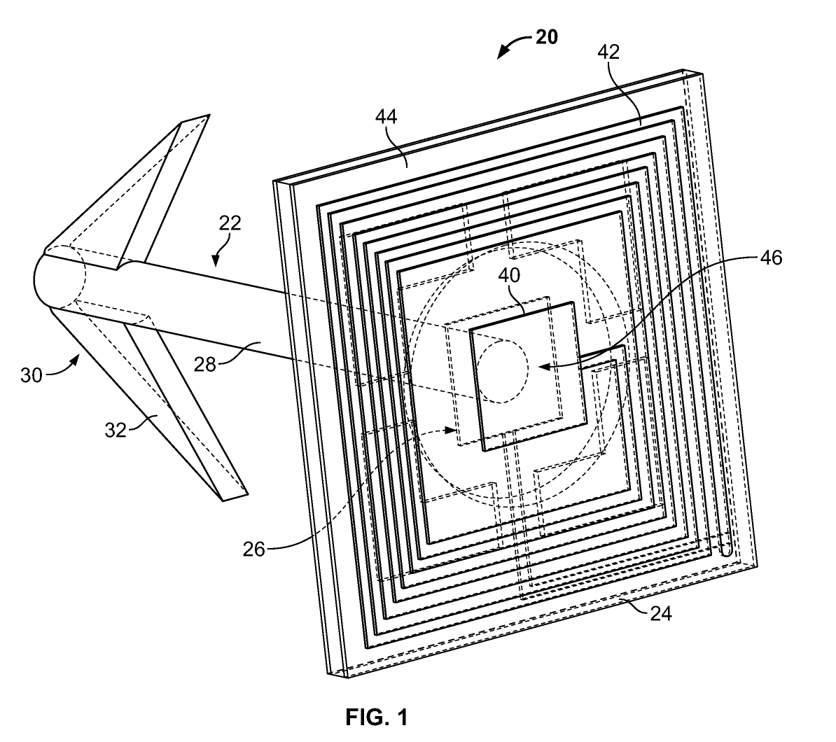

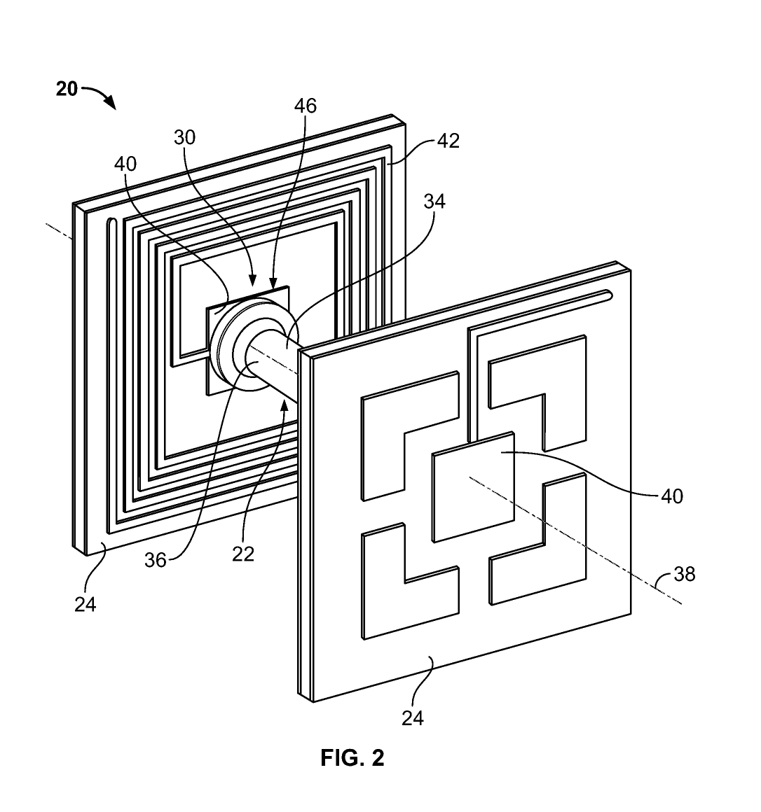

Referring to FIGS. 1 and 2, in one embodiment an implantable sensing unit 20 includes an anchoring mechanism 22 and at least one sensor 24 coupled to a first end 26 of anchoring mechanism 22. In one embodiment, sensor 24 is coupled to first end 26 using a suitable biocompatible adhesive including, without limitation, an acrylic-based adhesive, such as cyanoacrylate, an epoxy-based adhesive, a polyurethane-based adhesive, and/or a silicon-based adhesive, such as organopolysiloxane. The use of these adhesives promotes long-term use of implantable sensing unit 20, while preventing or limiting health risks to the patient. Additionally or alternatively, sensor 24 is coupled to first end 26 using a suitable mechanism and/or process known to those skilled in the art and guided by the teachings herein provided including, without limitation, a chemical bonding process, a heat bonding process, a soldering process, a suturing process using a non-absorbable suture, or an outer packaging material.

Sensor 24 senses a physical, chemical, and/or physiological parameter within a heart chamber. Sensor 24 may be a pressure sensor, an optical sensor, a biochemical sensor, a protein sensor, a motion sensor, an accelerometer, a gyroscope, a temperature sensor, a chemical sensor, a pH sensor, and/or a genetic sensor. In one embodiment, implantable sensing unit 20 includes one or more sensors 24 that are fabricated using a suitable microelectromechanical systems (MEMS) technology that utilizes a resonating frequency of an LC resonator. In alternative embodiments, implantable sensing unit 20 includes one or more sensors 24 that function as capacitive, inductive, piezoelectric or piezoresistive sensors. In alternative embodiments, implantable sensing unit 20 includes an ASIC having one or more sensors 24 integrally formed with the ASIC. In further alternative embodiments, one or more sensing units 20 are electrically connected or inductively coupled to an ASIC, wherein the ASIC may or may not include integrally formed sensors.

As shown in FIG. 1, anchoring mechanism 22 includes a post 28 defining first end 26 and a coupling member 30 configured to couple to tissue to facilitate implanting sensing unit 20 within a patient. In one embodiment, coupling member 30 includes a barbed member 32, as shown in FIG. 1, or a tethering member 34, as shown in FIG. 2, to facilitate coupling sensing unit 20 to tissue. Alternatively or in addition, coupling member 30 includes any suitable mechanism or component to facilitate coupling sensing unit 20 to tissue including, without limitation, a hook, a helical wire, and a screw. Coupling member 30 may be attached to a septum wall, a ventricle wall or atrium wall and is configured to counteract any inertial force and/or other forces generated during the fluid flow and/or movement of the heart wall during regular cardiac cycles.

As shown in FIG. 2, in one embodiment implantable sensing unit 20 includes a second sensor 24 coupled to a second end 36 of anchoring mechanism 22 opposing first end 26. Tethering member 34 couples sensors 24 and biases sensors 24 together. In a particular embodiment, tethering member 34 is fabricated of a suitable biocompatible material, such as a non-absorbable prolene material, having suitable elastic properties to provide a desired amount of recoverable strain at a relatively low stress level. In this embodiment, tethering member 34 is in constant tension to hold sensors 24 in compression against a septum wall, for example. Tethering member 34 is designed to exhibit a high elastic region allowing tethering member 34 to act as a spring applying a constant load based on a displacement strain. Tethering member 34 subsequently is allowed to return to an initial or original shape with a stress removed without permanent plastic deformation.

Further, tethering member 34 couples sensors 24 together such that a surface of each sensor 24 is positioned within a respective plane normal to a central axis 38 of tethering member 34, as shown in FIG. 2. For example, in one application, tethering member 34 is coupled to a first sensor 24, such as a pressure sensor, positioned within a left atrium of a patient's heart and a second sensor 24, such as a pressure sensor, positioned within a right atrium of the patient's heart such that respective longitudinal planes of sensors 24 lay normal to center axis 38 of tethering member 34. In this embodiment, an outer surface of sensor 24 lays generally parallel to the respective heart chamber wall. In a particular embodiment, sensor 24 is positioned about 0.5 mm to about 3.0 mm or, more specifically, about 1.0 mm to about 2.0 mm, from the respective heart chamber wall to prevent or limit cellular growth over the working components of sensor 24, described in greater detail below, which may degrade or weaken signals transmitted from sensor 24 to an external receiver (not shown). In this embodiment, sensors 24 resonate at different frequencies such that an external receiver may easily distinguish the signals from each sensor 24.

Referring further to FIGS. 1 and 2, in one embodiment sensor 24 includes a pressure sensor having a capacitor 40 and an inductor coil 42 set up in parallel and constructed on a structural substrate 44. Substrate 44 is fabricated to define a void 46 configured to receive components of capacitor 40 including, without limitation, a diaphragm with a capacitor plate which deflects in response to a changing environmental pressure within the heart chamber with sensing unit 20 affixed or coupled to the patient's target tissue. In alternative embodiments, substrate 44 is planar, and an additional layer is bonded to substrate 44 to define a diaphragm over a cavity. Sensor 24 may include any suitable sensor known to those skilled in art and guided by the teachings herein provided.

In one embodiment, sensor 24 is fabricated using a suitable microelectromechanical systems (MEMS) technology. In a particular embodiment, sensor 24 is fabricated using a MEMS technology that utilizes a resonating frequency of an LC Tank circuit or a suitable capacitive, inductive, piezoresistive or piezoelectric technology to measure pressure within the heart chamber. Sensor 24 is configured to facilitate transmission of data wirelessly to an external device, such as a user-controlled or handheld receiver. In a biomedical application, the signal is desirably transmitted through the patient's surrounding tissue without distorting or lowering a strength of the signal such that the signal is lost or undecipherable.

In a particular embodiment, sensor 24 includes a capacitance inductor circuit arranged in a parallel configuration to form an LC tank circuit. The LC tank circuit generates resonating frequency signals that are emitted from sensor 24 and transmitted to an at least partially external device, such as a patient signaling device, wherein the signals are processed and deciphered. For example, in one embodiment, the signals are transmitted to an implanted receiver, such as a subcutaneous antenna patch, and then wirelessly transmitted from the implanted receiver to an external receiver. Wireless communication in this manner may increase the wireless link distance allowed between the external reader and the implanted sensor. Based on the transmitted signals, the external device generates an output representative of a cardiac pressure within the respective heart chamber, for example. More specifically, in one embodiment, sensor 24 is configured to sense an internal pressure within the respective heart chamber and generate a signal representative of the internal pressure to facilitate measuring and/or monitoring cardiac blood pressure, for example. It should be apparent to those skilled in the art and guided by the teachings herein provided that sensor 24 may be fabricated using any suitable technology and/or process. In alternative embodiments, implantable sensing unit 20 includes a plurality of sensors 24 including a capacitive pressure sensing device, an inductive pressure sensing device, a piezoelectric pressure sensing device or a piezoresistive pressure sensing device.

In one embodiment, sensing unit 20 includes a suitable pressure sensor 24, which operates through a displacement of two capacitor plates that are connected in parallel to an inductor. As blood flows through the respective chamber, a pressure is induced on one or both capacitor plates. This pressure displaces the capacitor plate(s) and subsequently changes the capacitance value of sensing unit 20. The resonating frequency emitted from sensing unit 20 is a function of the inductance and capacitance values seen in the circuitry. Because the capacitance value of the circuitry changes with the changing internal pressure within the patient's heart chamber, the subsequently emitted resonating frequency will change with the changing internal pressure. This shift in the resonating frequency can be read through an external receiver unit and deciphered to generate an internal pressure reading within the patient's heart chamber. In alternative embodiments, spaced apart such that the displacement of the coils generates a change in capacitance. In alternative embodiments incorporating LC sensors, the inductance value of the sensor may change with pressure. It is within the scope of this disclosure that several sensing schemes may be employed to sense one of a variety of physiological parameters.

In one embodiment, sensor 24 is at least partially coated with at least one biocompatible material including, without limitation, one or more suitable biocompatible polymers such as a slow release polymer impregnated with an anti-metabolite inhibiting in-tissue growth. In a particular embodiment, at least a portion of sensor 24 is coated with a drug eluting material that prohibits in-tissue growth on sensor 24. In an alternative embodiment, at least a portion of sensor 24 is coated with a biocompatible material to promote in-tissue growth on sensor 24.

In one embodiment, sensor 24 is permanently affixed to barbed member 32, shown in FIG. 1, using a suitable biocompatible process that facilitates minimizing degradation over an extended period of time. Sensing unit 20 is subsequently implanted into the patient's targeted tissue by a practicing physician exerting a suitable piercing force transmitted generally normal to a center axis of post 28. The piercing force urges barbed member 32 to penetrate into or through the target tissue. Barbed member 32 facilitates implantation of sensing unit 20 into the target tissue. Further, barbed member 32 provides a resistive force when sensing unit 20 experiences a pulling force to facilitate preventing sensing unit 20 from undesirably pulling free from the target tissue as a result of movement of the heart and/or blood flow through the heart, for example.

Referring to FIG. 2, sensing unit 20, including two sensors 24 coupled to opposing ends of tethering member 34, is implanted within a patient's cardiac system. In a particular application, tethering member 34 is positioned through a patient's septum wall, which separates the right atrium and the left atrium of the heart, for example. A first sensor 24 is positioned within the right atrium and a second sensor 24 is positioned within the left atrium of the patient's heart. With each sensor 24 properly positioned within respective atrium, tethering member 34 provides a suitable biasing force to bias sensors 24 together and retain sensors 24 positioned as desired within the respective atrium.

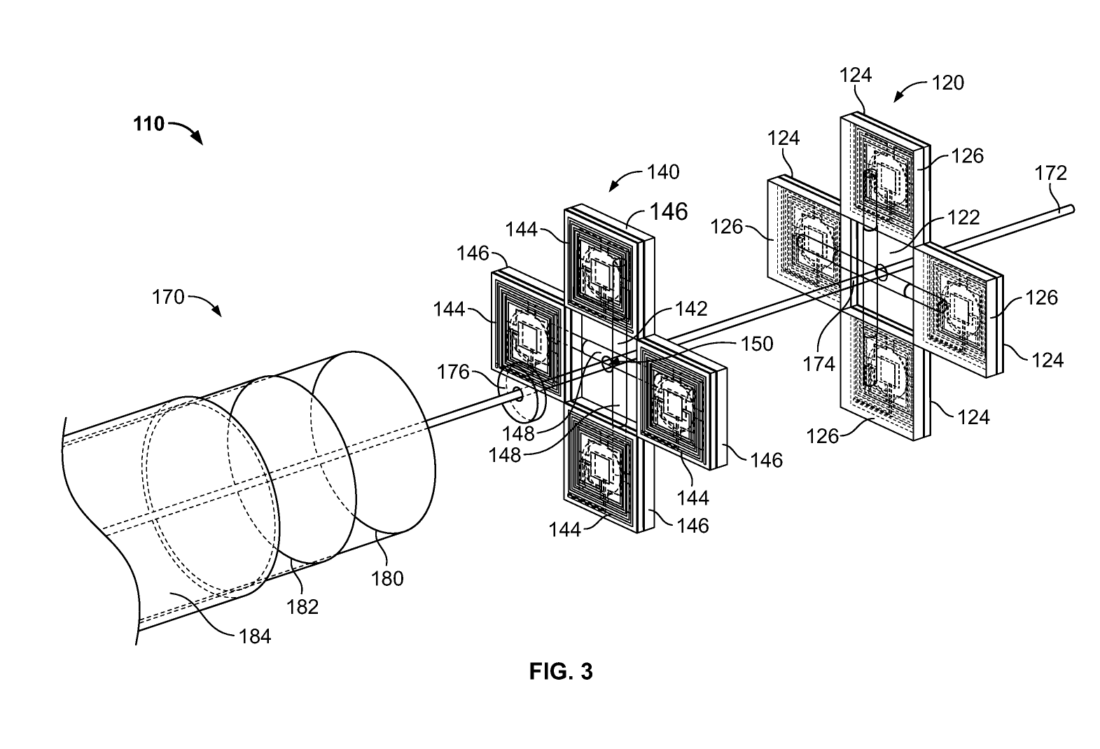

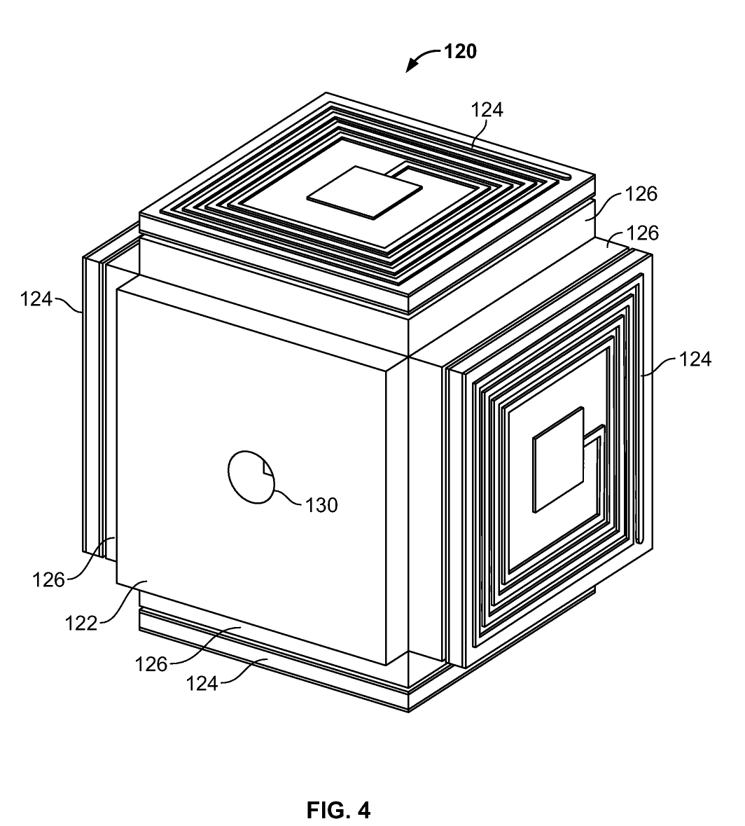

Referring to FIGS. 3-10, in one embodiment an implantable medical device 110 includes a first sensor array 120 having a main portion 122 and at least one rigid sensor 124 movably coupled to main portion 122. As used herein, the term “rigid” refers to an inability of the sensor to bend greater than about 90° without fracture or plastic deformation. In a particular embodiment, rigid sensor 124 is fabricated from a thin glass or fused silica material and, as such, may be flexible to a certain extent (compared to sensors made of a thick material). In an alternative embodiment, at least a portion of sensor 124 is flexible or bendable. As shown in FIGS. 3-10, in the exemplary embodiment implantable medical device 110 includes a plurality of rigid sensors 124 each movably coupled to main portion 122. In this embodiment, main portion 122 is centrally located and each sensor 124 extends radially outwardly from main portion 122. Each sensor 124 is pivotally and/or rotationally movable with respect to main portion 122 such that first sensor array 120 is movable between a closed or collapsed configuration, as shown in FIG. 4, and a deployed configuration, as shown in FIG. 5. In one embodiment, one or more sensors 124 sense the same parameter, e.g. pressure, and one or more sensors 124 resonate at the same frequency. In this embodiment, the signals received from an external receiver may sum the signals of one or more sensors 124 such that the signal received at the external receiver is greater than that received from one sensor 124 alone. In an alternative embodiment, one or more sensors 124 sense a different parameter, resonate at a different frequency, and/or have a signal that may be readily differentiated from other sensors 124 when the signal is received by the receiver.

In one embodiment, first sensor array 120 includes one or more substrate portions 126 movably coupled to main portion 122. As shown in FIGS. 3-7, main portion 122 defines a center portion of first sensor array 120 and each substrate portion 126 extends radially outwardly from main portion 122. Substrate portion 126 is fabricated of any suitable biocompatible material including, without limitation, a suitable polymer, ceramic, metal (such as gold, silver, titanium), alloy (such as stainless steel), composite, silicon, fused silica, or shape memory material (such as a Nitinol material).

In a particular embodiment, each substrate portion 126 is pivotally and/or rotationally coupled to or with respect to main portion 122 at or near an attachment point or line. In the exemplary embodiment, first sensor array 120 includes a plurality of substrate portions 126 movably coupled to first main portion 122. One or more sensors 124 are coupled to a corresponding substrate portion 126, as shown in FIGS. 3-7. In one embodiment, main portion 122 and/or substrate portions 126 include projections, such as microneedles formed using a suitable MEMS technology, to facilitate retaining first sensor array 120 properly positioned within the heart chamber.

Referring further to FIG. 5, each substrate portion 126, in one embodiment, is movably coupled to main portion 122 using a bendable strut 128. In a particular embodiment, strut 128 is fabricated at least partially from a material having shape memory properties such that first sensor array 120 is movable between the collapsed configuration, shown in FIG. 4, and the deployed configuration, shown in FIG. 5, as desired. Strut 128 is initially in a bent configuration and moves towards a straight configuration once heated, for example. In the deployed configuration, sensor 124 and/or corresponding substrate portion 126 is preferably coplanar with main portion 122. Suitable materials for fabricating strut 128 include, without limitation, Nitinol and other known shape memory alloys (SMA) having properties that develop a shape memory effect (SME), which allows the material to return to an initial configuration after a force applied to the material to shape, stretch, compress and/or deform the material is removed. In a further embodiment, strut 128 is fabricated from a thermally treated metal alloy (TMA) including, without limitation, nickel titanium, beta titanium, copper nickel titanium and any combination thereof. In an alternative embodiment, strut 128 is fabricated at least partially from a suitable polymeric material. It should be apparent to those skilled in the art and guided by the teachings herein provided that strut 128 may be fabricated using any suitable biocompatible material preferably, but not necessarily, having suitable shape memory properties. As shown further in FIGS. 4 and 5, main portion 122 defines an aperture 130 therethrough to facilitate implanting implantable medical device 110 within the patient, as described in greater detail below.

It should be apparent to those skilled in the art and guided by the teachings herein provided that each substrate portion 126 may be movably coupled to or integrated with main portion 122 using any suitable coupling mechanism and/or any suitable material. For example, in alternative embodiments each substrate portion 126 may be fabricated of a suitable shape memory material integrated with or coupled to main portion 122 and movable with respect to main portion 122 such that first sensor array 120 is movable between the collapsed configuration and the deployed configuration. Alternatively, each substrate portion 126 may be mechanically coupled, such as hingedly coupled, to main portion 122 such that first sensor array 120 is movable between the collapsed configuration and the deployed configuration. In a further alternative embodiment, each substrate portion 126 is coupled to main portion 122 during fabrication using any suitable process, such as an adhesion process that includes a suitable biocompatible adhesive including, without limitation, an acrylic-based adhesive, such as cyanoacrylate, an epoxy-based adhesive, a polyurethane-based adhesive, and/or a silicon-based adhesive, such as organopolysiloxane. The use of these adhesives promotes long-term use of implantable medical device 110, while preventing or limiting health risks to the patient.

A second sensor array 140 is operatively coupled to first sensor array 120. In the exemplary embodiment, second sensor array 140 is similar to first sensor array 120. Second sensor array 140 includes a main portion 142 and at least one rigid sensor 144 movably coupled to main portion 142. In the exemplary embodiment, a plurality of rigid sensors 144 are movably coupled to main portion 142. In this embodiment, main portion 142 is centrally located and each sensor 144 extends radially outwardly from main portion 142.

In one embodiment, second sensor array 140 includes one or more substrate portions 146 movably coupled to main portion 142. Main portion 142 defines a center portion of second sensor array 140 and each substrate portion 146 extends radially outwardly from main portion 142. Substrate portion 146 is fabricated of any suitable biocompatible material having sufficient flexibility including the materials described above in reference to substrate portion 126. In a particular embodiment, each substrate portion 146 is pivotally and/or rotationally coupled to or with respect to main portion 142 at or near an attachment point or line. In the exemplary embodiment, second sensor array 140 includes a plurality of substrate portions 146 movably coupled to main portion 142. One or more sensors 144 are coupled to a corresponding substrate portion 146, as shown in FIG. 3 for example.

Each substrate portion 146, in one embodiment, is movably coupled to main portion 142 using a bendable strut 148, as shown in FIG. 3. Strut 148 is similar to strut 128 described above such that second sensor array 140 is movable between the collapsed configuration and the deployed configuration, as desired. As shown in FIG. 3, main portion 142 defines an aperture 150 therethrough to facilitate implanting implantable medical device 110 within the patient, as described in greater detail below.

Each sensor 124 and each sensor 144 senses a physical, chemical, and/or physiological parameter within a respective heart chamber. Sensor 124 and sensor 144 may be a pressure sensor, an optical sensor, a biochemical sensor, a protein sensor, a motion sensor, an accelerometer, a gyroscope, a temperature sensor, a chemical sensor, a pII sensor, and/or a genetic sensor. In one embodiment, implantable medical device 110 includes one or more sensors 124 and/or one or more sensors 144 that are fabricated using a suitable microelectromechanical systems (MEMS) technology that utilizes a resonating frequency of an LC resonator. In alternative embodiments, implantable medical device 110 includes one or more sensors 124 and/or one or more sensors 144 that function as capacitive, inductive, piezoelectric or piezoresistive sensors. In alternative embodiments, implantable medical device 110 includes an ASIC having one or more sensors integrally formed with the ASIC. In an alternative embodiment, one or more sensors may be electrically connected or inductively coupled to an ASIC, wherein the ASIC may or may not include integrally formed sensors. First sensor array 120 and second sensor array 140 are configured to facilitate obtaining data for cardiac pressure analysis, temperature analysis, blood chemical analysis, blood osmolar analysis, and/or cellular count analysis. First sensor array 120 and second sensor array 140 generate and ultimately transmit signals representative of measurement data wirelessly to an external receiver.

In one embodiment, one or more sensors 124 and/or one or more sensor 144 are fabricated using a suitable microelectromechanical systems (MEMS) technology. In a particular embodiment, sensors 124 and sensors 144 are fabricated using a MEMS technology that utilizes a resonating frequency of an LC Tank circuit or a suitable capacitive, inductive, piezoresistive, or piezoelectric technology to measure pressure within the heart chamber. In a particular embodiment, sensors 224 and sensors 244 include a hermetically sealed chamber such that the sensor signal does not appreciably drift with time due to molecular diffusion. Sensors 124 and sensors 144 are configured to facilitate transmission of data wirelessly to an external device, such as a user-controlled or handheld receiver. In a biomedical application, the signal is desirably transmitted through the patient's surrounding tissue without distorting or lowering a strength of the signal such that the signal is lost or undecipherable.

In a particular embodiment, sensors 124 and sensors 144 include a capacitance inductor circuit arranged in a parallel configuration to form an LC tank circuit. The LC tank circuit generates resonating frequency signals that are emitted from sensors 124 and/or sensors 144 and transmitted to an at least partially external device, such as a patient signaling device, wherein the signals are processed and deciphered. In one embodiment, a compatible telemetry patch is external to the skin, such as adhered to an outer skin surface of the patient, or subcutaneous. In this embodiment, the signals may be processed and/or deciphered at the telemetry patch and/or the external device. Based on the transmitted signals, the external device generates an output representative of a cardiac pressure within the respective heart chamber, for example. More specifically, in one embodiment, sensors 124 and sensors 144 sense an internal pressure within the respective heart chamber and generate a signal representative of the internal pressure to facilitate measuring and/or monitoring cardiac blood pressure, for example. It should be apparent to those skilled in the art and guided by the teachings herein provided that sensors 124 and sensors 144 may be fabricated using any suitable technology and/or process. In alternative embodiments, implantable medical device 110 includes a plurality of sensors 124 and/or a plurality of sensors 144 including a capacitive pressure sensing device, an inductive pressure sensing device, a piezoelectric pressure sensing device or a piezoresistive pressure sensing device. In alternative embodiments, implantable medical device 110 includes an ASIC having one or more sensors integrally formed with the ASIC. In alternative embodiments, implantable medical device 110 includes one or more sensors electrically connected or inductively coupled to an ASIC, wherein the ASIC may or may not include integrally formed sensors.

In one embodiment, medical device 110 includes suitable sensors 124 and sensors 144, which operate through a displacement of two capacitor plates that are connected in parallel to an inductor. Medical device 110 is implanted within a patient's heart chamber. The capacitor plates are located on opposite sides of a hermetic chamber. At least one portion of the hermetic chamber is responsive to an externally applied pressure. One or more of the capacitor plates may reside inside the hermetic chamber or outside the hermetic chamber. The capacitor plates are operatively coupled to at least one portion of the hermetic chamber that is responsive to an externally applied pressure. As blood flows through the respective heart chamber, a pressure is induced on one or both capacitor plates. This pressure displaces the capacitor plate(s) and subsequently changes the capacitance value of medical device 110. The resonating frequency emitted from medical device 110 is a function of the inductance and capacitance values seen in the circuitry. Because the capacitance values of the circuitry changes with the changing internal pressure within the patient's heart chamber, the subsequently emitted resonating frequency will change with the changing internal pressure. This shift in the resonating frequency can be read through an external receiver unit and deciphered to generate an internal pressure reading within the patient's heart chamber. In a particular embodiment, the sensing diaphragm of the sensor is sufficiently stiff so that when the diaphragm is covered with approximately 300 micrometers of tissue, the diaphragm stiffness with the tissue is within about 5% or, more specifically, within about 1%, of the diaphragm stiffness without the tissue. Further, the volume between the capacitor plates and/or inductor is hermetically sealed. An acceptable hermetic seal includes sufficiently low porosity materials so that transfer of molecules into and/or out of the hermetic chamber does not cause drift of the sensor by approximately more than 1 mm Hg per year.

In one embodiment, sensors 124 and sensors 144 are at least partially coated with at least one biocompatible material including, without limitation, one or more suitable biocompatible polymers such as a slow release polymer impregnated with an anti-metabolite inhibiting in-tissue growth. In a particular embodiment, at least a portion of sensors 124 and/or sensors 144 are coated with a drug eluting material that prohibits in-tissue growth on sensors 124 and sensors 144. In an alternative embodiment, at least a portion of sensors 124 and/or sensors 144 are coated with a biocompatible material to promote in-tissue growth on sensors 124 and/or sensors 144.

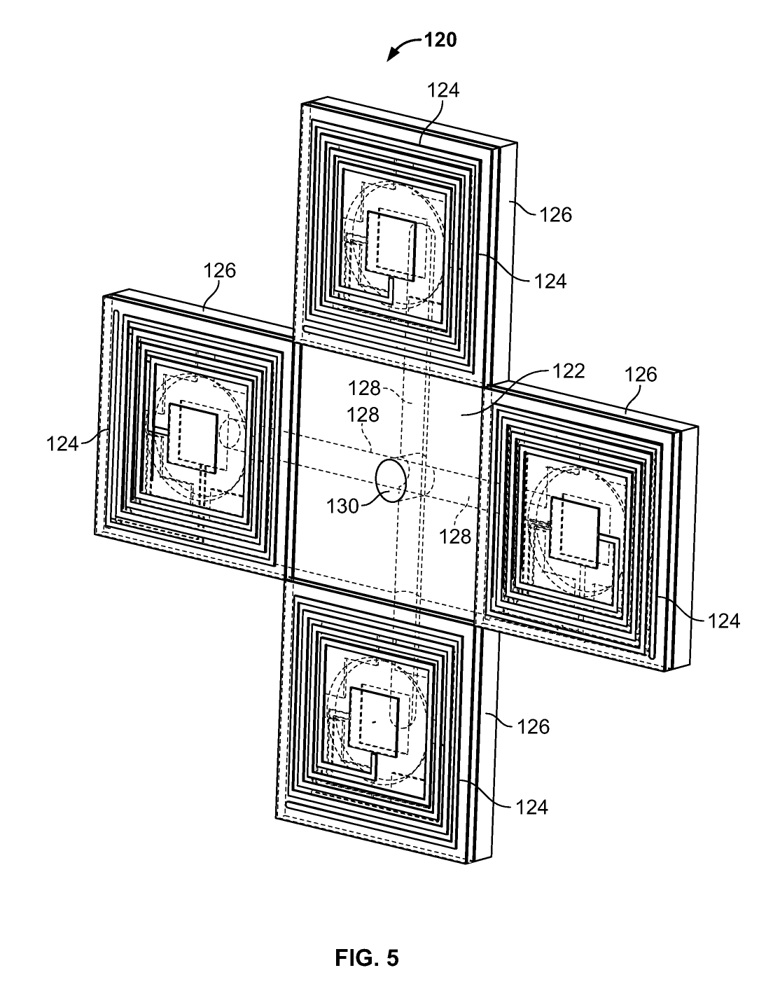

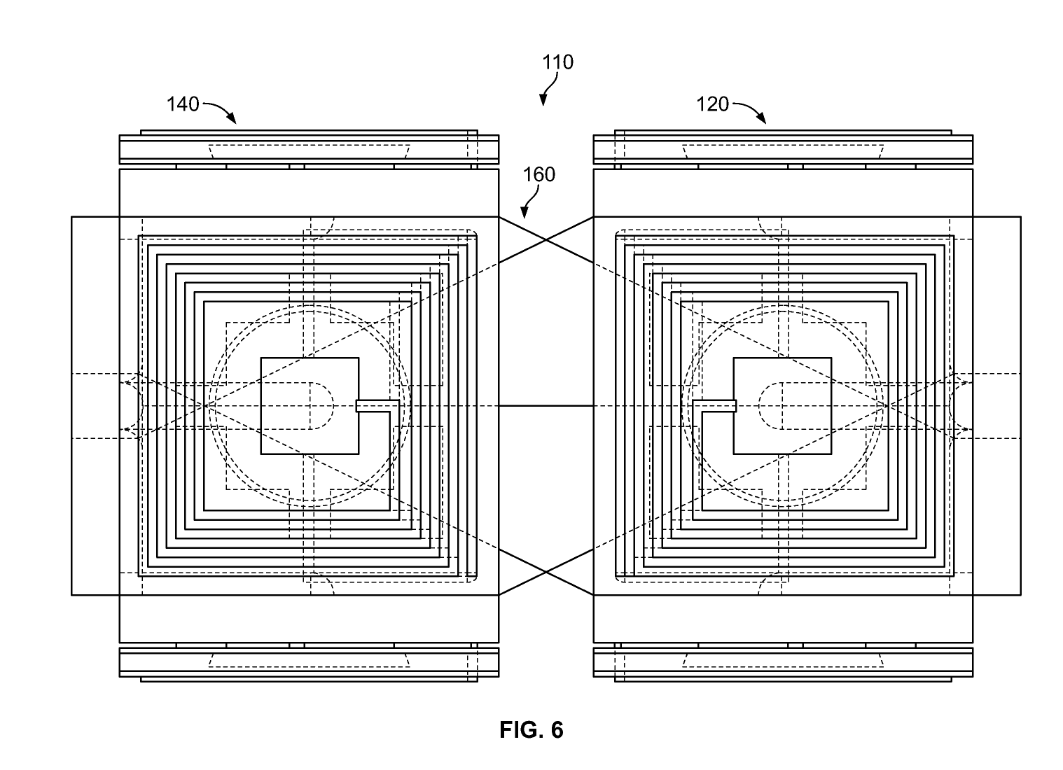

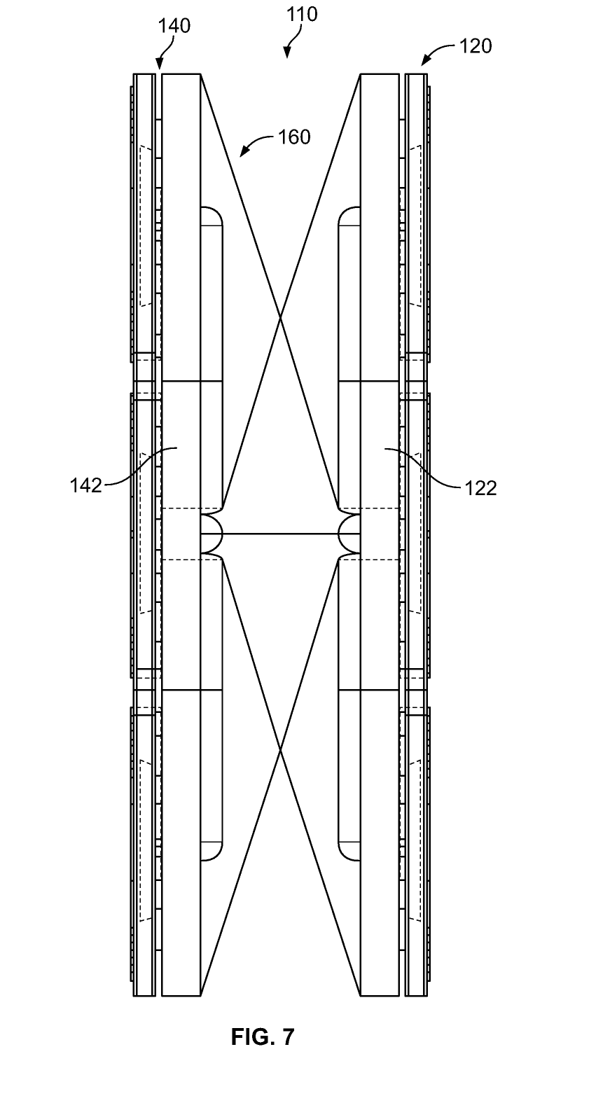

Referring further to FIGS. 6 and 7, a coupler, such as a tethering mechanism 160, operatively couples first sensor array 120 to second sensor array 140 to facilitate moving first sensor array 120 and second sensor array 140 between the collapsed configuration and the deployed configuration. Further, in the deployed configuration tethering mechanism 160 urges first sensor array 120 towards second sensor array 140 to facilitate retaining medical device 110 properly positioned within the patient's heart. In one embodiment, tethering mechanism 160 is coupled to main portion 122 of first sensor array 120 and main portion 142 of second sensor array 140. Alternatively or in addition, tethering mechanism 160 is coupled to one or more substrate portions 126 and/or one or more substrate portions 146. Upon deployment of implantable medical device 110, tethering mechanism 160 urges opposing main portion 122 and main portion 142 towards respective surfaces of the tissue wall.

Referring further to FIGS. 8-10, in alternative embodiments, first sensor array 120, shown in FIGS. 8-10, and second sensor array 140 of implantable medical device 110 may have any suitable shape and/or configuration including any suitable number of sensors 124 or 144, respectively. FIG. 8 shows first sensor array 120 in a deployed configuration. FIG. 9 shows first sensor array 120 in a partially closed or collapsed configuration. FIG. 10 shows first sensor array 120 in a fully closed or collapsed configuration.

Further, main portion 122 and/or main portion 142 may include a plurality of movably coupled segments, such as first main portion segment 162 and second main portion segment 164 as shown in FIGS. 8-10, to further facilitate moving respective first sensor array 120 between the collapsed configuration, as shown in FIG. 10, and the deployed configuration, as shown in FIG. 8. Referring further to FIG. 10, in one embodiment first sensor array 120 and/or second sensor array 140 is foldable to the collapsed configuration for insertion into the patient. In the folded position, each substrate portion 126 or each substrate portion 146 is folded to contact main portion 122 or main portion 142, respectively.

Referring again to FIG. 3, implantable medical device 110 is deployed at a target tissue site using a suitable catheter system 170. First sensor array 120 and second sensor array 140 are positioned about a guide wire 172. More specifically, guide wire 172 is positioned through aperture 130 defined through main portion 122 and through aperture 150 defined through main portion 142. In one embodiment, a connecter 174 is positioned between first sensor array 120 and second sensor array 140 to facilitate coupling first sensor array 120 to second sensor array 140. Additionally, a locking plate 176 is positioned on an opposing side of second sensor array 140 to couple second sensor array 140 to connecter 174. Catheter system 170 includes a small sheath 180, a medium sheath 182 positioned about small sheath 180 and a large sheath 184 positioned about medium sheath 182 to facilitate delivering and deploying implantable medical device 110 at the target tissue site. In a particular embodiment, small sheath 180 has a five to seven French diameter, medium sheath 182 has a eight to ten French diameter, and large sheath 184 has a nine to twelve French diameter.

In this embodiment, an outer surface of sensor 124 and an outer surface of sensor 144 lay generally parallel to the respective heart chamber wall. In a particular embodiment, sensor 124 and sensor 144 are positioned about 0.5 mm to about 3.0 mm or, more specifically, about 1.0 mm to about 2.0 mm, from the respective heart chamber wall to prevent or limit cellular growth over the working components of sensor 124 and sensor 144, which may degrade or weaken signals transmitted from sensor 124 and sensor 144 to an external receiver (not shown).

With first sensor array 120 and second sensor array 140 in the collapsed configuration, second sensor array 140 is positioned between medium sheath 182 and small sheath 180 to retain second sensor array 140 in the collapsed configuration and first sensor array 120 is positioned between large sheath 184 and medium sheath 182 to retain first sensor array 120 in the collapsed configuration. Implantable medical device 110 is inserted into the patient's femoral artery at a puncture site. Guide wire 172 directs implantable medical device 110 into the right atrium of the patient. Once the right atrium is identified, it is acceded and the atrium septum is identified. If the septum is intact, a puncture is formed through the septum using a suitable technique, such as a Brockenberg Needle technique and guide wire 172 is inserted through the puncture.

With implantable medical device 110 positioned at the target tissue site, such as positioned within a hole defined through a septum wall, large sheath 184 is moved distally with respect to medium sheath 182 to release first sensor array 120 within a first chamber of the heart, i.e., the left atrium. Within the left atrium, first sensor array 120 is deployed and moves from the collapsed configuration, as shown in FIG. 4, to the deployed configuration, as shown in FIG. 5. Medium sheath 182 is then moved distally with respect to small sheath 180 to release second sensor array 140 within a second chamber of the heart, i.e., the right atrium. Within the right atrium, second sensor array 140 is deployed and moves from the collapsed configuration to the deployed configuration. Locking plate 176 is then moved proximally along guide wire 172 to urge second sensor array 140 towards first sensor array 120 and secure second sensor array 140 to connecter 174 to retain implantable medical device 10 properly positioned within the septum wall with first sensor array 120 properly positioned with respect to a surface of the septum wall within the left atrium and second sensor array 140 properly positioned with respect to a surface of the septum wall within the right atrium.

In one embodiment, implantable medical device 110 is inserted through the septum with first sensor array 120 and second sensor array 140 positioned on opposing sides of the septum wall. The shape memory material deforms to change a microstructure from a martensite structure to an austenite structure. This deformation of the shape memory material urges the shape memory material to return to an original configuration effectively moving or urging the sensor arrays toward each other. This deformation of the shape memory material may be related to the environmental temperature in which the material resides and per design will change the microstructure from martensite to austenite or vise-versa at a temperature range of about room temperature (70° F.) to about body temperature of a living being (98.7° F.). This temperature range is dependent on the mass fraction of the elements forming the shape memory material.

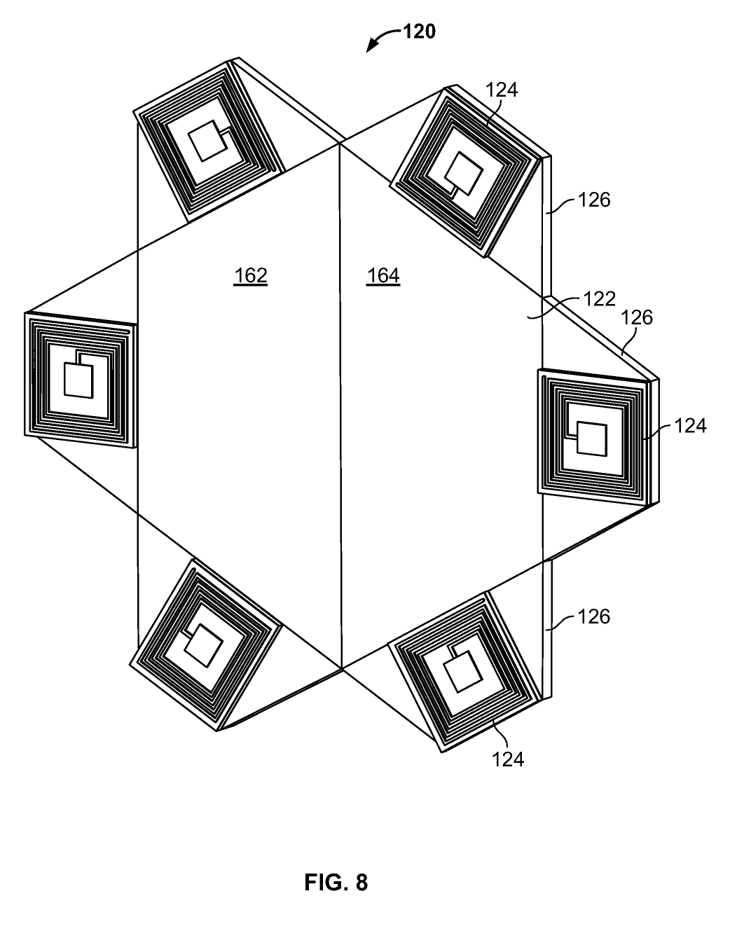

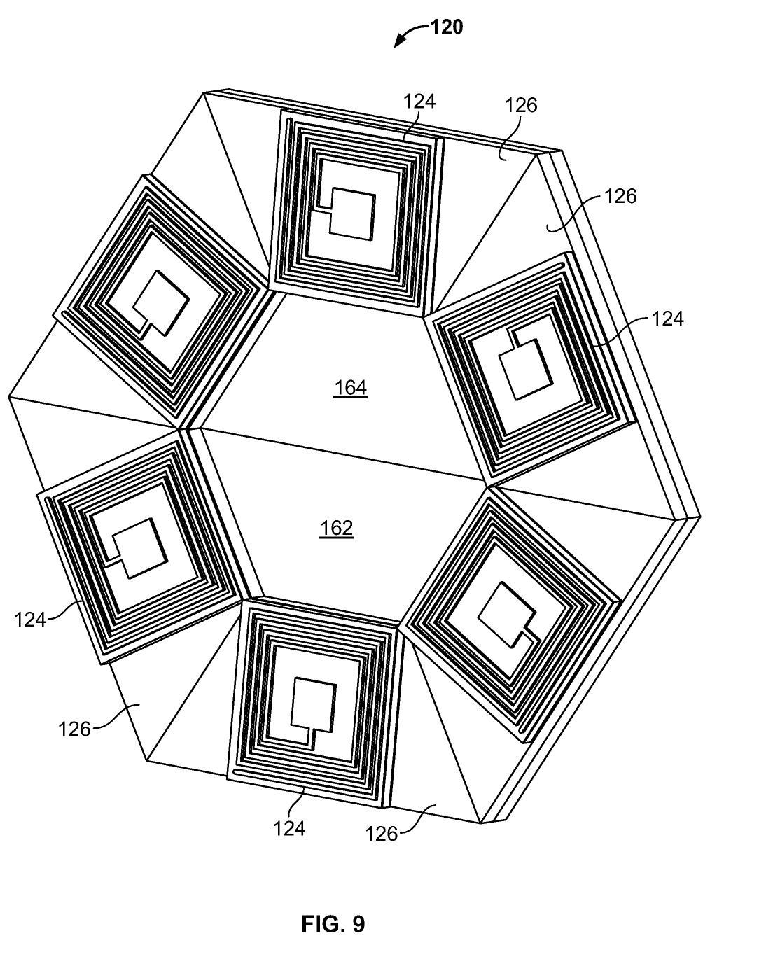

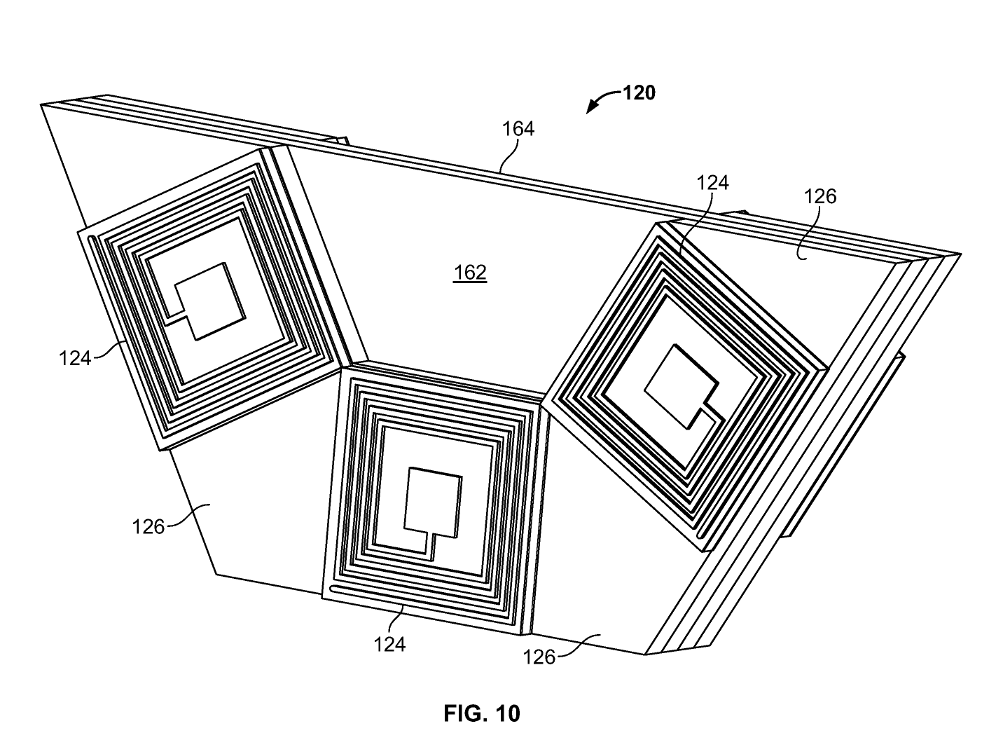

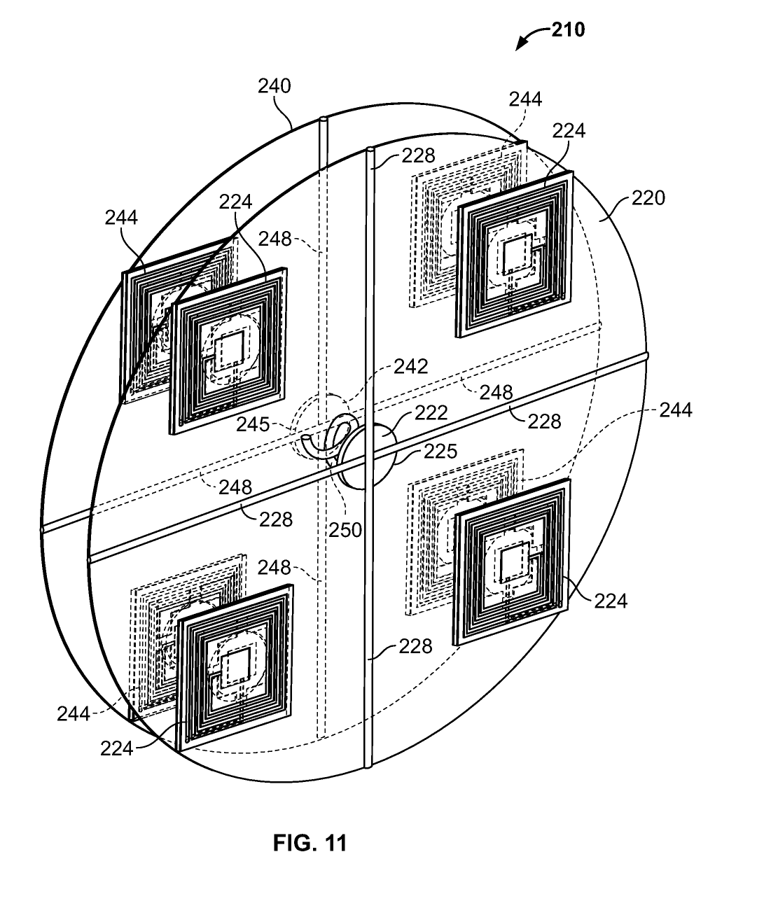

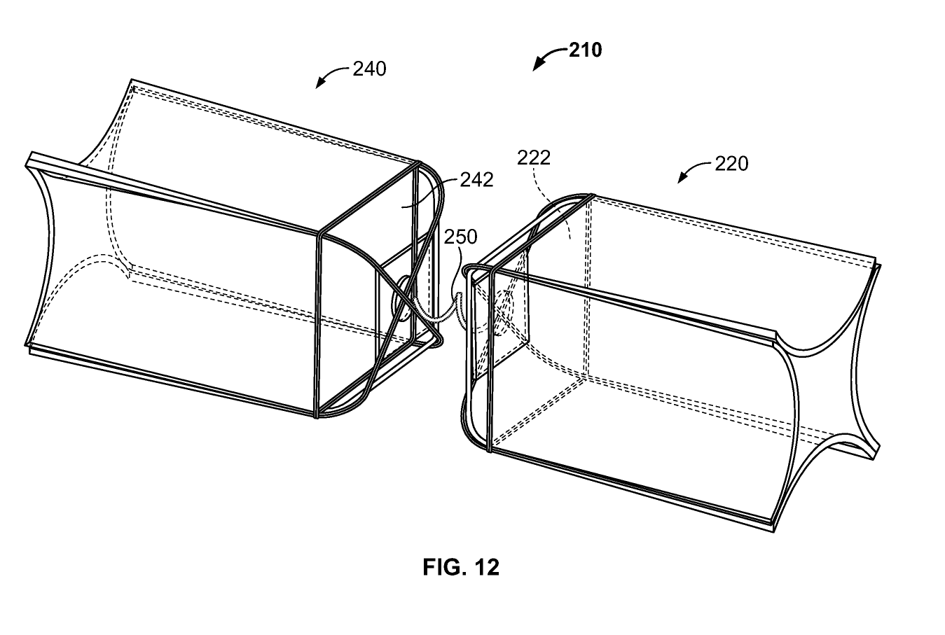

Referring to FIGS. 11 and 12, in one embodiment an implantable medical device 210 includes a flexible, biocompatible first substrate 220 having a center portion 222 and at least one rigid sensor 224 coupled to first substrate 220. In certain embodiments, first substrate 220 is made of a flexible material and/or first substrate 220 includes a coupling mechanism that is flexible such that first substrate 220 is flexible. In a particular embodiment, center portion 222 includes a rigid central retainer 225 coupled to first substrate 220. First substrate 220 is fabricated using any suitable biocompatible material having sufficient flexibility including, without limitation, a suitable polymer, ceramic, metal, alloy, composite or silicon material. In an alternative embodiment, at least a portion of sensor 224 is flexible or bendable. As shown in FIG. 11, implantable medical device 210 includes a plurality of rigid sensors 224 each coupled to first substrate 220. In this embodiment, each sensor 224 is positioned radially outwardly from center portion 222.

First substrate 220 is movable between a closed or collapsed configuration, as shown in FIG. 12, and a deployed configuration, as shown in FIG. 11. One or more bendable struts 228 are coupled to first substrate 220. Each strut 228 extends radially outwardly from center portion 222 defined by said first substrate 220. In a particular embodiment, strut 228 is fabricated at least partially from a material having shape memory properties such that first substrate 220 is movable between the collapsed configuration and the deployed configuration, as desired. Strut 228 is initially in a bent configuration to urge or bias first substrate 220 towards the collapsed configuration, as shown in FIG. 12, and moves towards a straight configuration, as shown in FIG. 11, upon deployment of implantable medical device 210 at a target site to urge or bias first substrate 220 towards the deployed configuration. In the deployed configuration, each sensor 224 is preferably coplanar with center portion 222. Suitable materials for strut 228 include, without limitation, Nitinol and other known shape memory alloys (SMA) having properties that develop a shape memory effect (SME), which allows the material to return to an initial configuration after a force applied to the material to shape, stretch, compress and/or deform the material is removed. In a further embodiment, strut 228 is fabricated from a thermally treated metal alloy (TMA) including, without limitation, nickel titanium, beta titanium, copper nickel titanium and any combination thereof. In an alternative embodiment, strut 228 is fabricated at least partially from a suitable polymeric material. It should be apparent to those skilled in the art and guided by the teachings herein provided that strut 228 may be fabricated using any suitable biocompatible material preferably, but not necessarily, having suitable shape memory properties.

Implantable medical device 210 also includes a flexible, biocompatible second substrate 240 having a center portion 242 operatively coupled to center portion 222 of first substrate 220. In certain embodiments, second substrate 240 is made of a flexible material and/or second substrate 240 includes a coupling mechanism that is flexible such that second substrate 240 is flexible. In the exemplary embodiment, second substrate 240 is similar to first substrate 220. Second substrate 240 is fabricated of any suitable biocompatible material having sufficient flexibility including materials described above in reference to first substrate 220. In one embodiment, at least one rigid sensor 244 is coupled to second substrate 240. In an alternative embodiment, at least a portion of sensor 244 is flexible or bendable. As shown in FIG. 11, implantable medical device 210 includes a plurality of rigid sensors 244 each coupled to second substrate 240. In this embodiment, each sensor 244 is positioned radially outwardly from center portion 242. In a particular embodiment, center portion 242 includes a rigid central retainer 245 coupled to second substrate 240.

Second substrate 240 is movable between a closed or collapsed configuration, as shown in FIG. 12, and a deployed configuration, as shown in FIG. 11. Each strut 228 extends radially outwardly from center portion 222 defined by said first substrate 220. One or more bendable struts 248 are coupled to second substrate 240. In a particular embodiment, strut 248 is fabricated at least partially from a material having shape memory properties such that second substrate 240 is movable between the collapsed configuration and the deployed configuration, as desired. Strut 248 is initially in a bent configuration, as shown in FIG. 12, to urge or bias second substrate 240 towards the collapsed configuration and moves towards a straight configuration, as shown in FIG. 11, upon deployment of implantable medical device 210 at a target site to urge or bias second substrate 240 towards the deployed configuration. In the deployed configuration, sensor 244 is preferably coplanar with center portion 242. Suitable materials for strut 248 include, without limitation, materials described above in reference to strut 228. In one embodiment, strut 248 is similar to strut 228 described above such that second substrate 240 is movable between the collapsed configuration and the deployed configuration, as desired.

Each sensor 224 and each sensor 244 senses a physical, chemical, and/or physiological parameter within a respective heart chamber. Sensor 224 and sensor 244 may be a pressure sensor, an optical sensor, a biochemical sensor, a protein sensor, a motion sensor, an accelerometer, a gyroscope, a temperature sensor, a chemical sensor, a pH sensor, and/or a genetic sensor. In one embodiment, implantable medical device 210 includes one or more sensors 224 and/or one or more sensors 244 that are fabricated using a suitable microelectromechanical systems (MEMS) technology that utilizes a resonating frequency of an LC resonator. In alternative embodiments, implantable medical device 210 includes one or more sensors 224 and/or one or more sensors 244 that function as capacitive, inductive, piezoelectric or piezoresistive sensors. In alternative embodiments, implantable medical device 210 includes an ASIC having one or more sensors integrally formed with the ASIC. In alternative embodiments, implantable medical device 210 includes one or more sensors that are electrically connected or inductively coupled to an ASIC, wherein the ASIC may or may not include integrally formed sensors.

First substrate 220 and second substrate 240 are configured to facilitate obtaining data for cardiac pressure analysis, temperature analysis, blood chemical analysis, blood osmolar analysis, and/or cellular count analysis. First substrate 220 and second substrate 240 generate and ultimately transmit signals representative of measurement data wirelessly to an external receiver (not shown).

In one embodiment, one or more sensors 224 and/or one or more sensor 244 are fabricated using a suitable microelectromechanical systems (MEMS) technology. In a particular embodiment, sensors 224 and sensors 244 are fabricated using a MEMS technology that utilizes a resonating frequency of an LC Tank circuit or a suitable capacitive, inductive, piezoresistive, or piezoelectric technology to measure pressure within the respective heart chamber. In a particular embodiment, sensors 224 and sensors 244 include a hermetically sealed chamber such that the sensor signal does not appreciably drift with time due to molecular diffusion. Sensors 224 and sensors 244 are configured to facilitate transmission of data wirelessly to an external device, such as a user-controlled or handheld receiver. In a biomedical application, the signal is desirably transmitted through the patient's surrounding tissue without distorting or lowering a strength of the signal such that the signal is lost or undecipherable.

In a particular embodiment sensors 224 and sensors 244 include a capacitance inductor circuit arranged in a parallel configuration to form an LC tank circuit. The LC tank circuit generates resonating frequency signals that are emitted from sensors 224 and/or sensors 244 and transmitted to an at least partially external device, such as a patient signaling device, wherein the signals are processed and deciphered. Based on the transmitted signals, the external device generates an output representative of a cardiac pressure within the respective heart chamber, for example. More specifically, in one embodiment, sensors 224 and sensors 244 sense an internal pressure within the respective heart chamber and generate a signal representative of the internal pressure to facilitate measuring and/or monitoring cardiac blood pressure, for example. It should be apparent to those skilled in the art and guided by the teachings herein provided that sensors 224 and sensors 244 may be fabricated using any suitable technology and/or process. In alternative embodiments, implantable medical device 210 includes a plurality of sensors 224 and/or a plurality of sensors 244 including a capacitive pressure sensing device, an inductive pressure sensing device, a piezoelectric pressure sensing device or a piezoresistive pressure sensing device.

In one embodiment, medical device 210 includes suitable sensors 224 and sensors 244, which operate through a displacement of two capacitor plates that are connected in parallel to an inductor. Medical device 210 is implanted within a patient's heart chamber. The capacitor plates are located on opposite sides of a hermetic chamber. At least one portion of the hermetic chamber is responsive to an externally applied pressure. One or more of the capacitor plates may reside inside the hermetic chamber or outside the hermetic chamber. The capacitor plates are operatively coupled to at least one portion of the hermetic chamber that is responsive to an externally applied pressure. As blood flows through the respective heart chamber, a pressure is induced on one or both capacitor plates. This pressure displaces the capacitor plate(s) and subsequently changes the capacitance value of medical device 210. The resonating frequency emitted from medical device 210 is a function of the inductance and capacitance values seen in the circuitry. Because the capacitance values of the circuitry changes with the changing internal pressure within the patient's heart chamber, the subsequently emitted resonating frequency will change with the changing internal pressure. This shift in the resonating frequency can be read through an external receiver unit and deciphered to generate an internal pressure reading within the patient's heart chamber. In a particular embodiment, the sensing diaphragm of the sensor is sufficiently stiff so that when the diaphragm is covered with approximately 300 micrometers of tissue, the diaphragm stiffness with the tissue is within about 5% or, more specifically, within about 1%, of the diaphragm stiffness without the tissue. Further, the volume between the capacitor plates and/or inductor is hermetically sealed. An acceptable hermetic seal includes sufficiently low porosity materials so that transfer of molecules into and/or out of the hermetic chamber does not cause drift of the sensor by approximately more than 1 mm Hg per year.

In one embodiment, sensors 224 and sensors 244 are at least partially coated with at least one biocompatible material including, without limitation, one or more suitable biocompatible polymers such as a slow release polymer impregnated with an anti-metabolite inhibiting in-tissue growth. In a particular embodiment, at least a portion of sensors 224 and sensors 244 are coated with a drug eluting material that prohibits in-tissue growth on sensors 224 and sensors 244. In an alternative embodiment, at least a portion of sensors 224 and/or sensors 244 are coated with a biocompatible material to promote in-tissue growth on sensors 224 and/or sensors 244.

In one embodiment, a connecting member 250 is coupled at a first end to first substrate 220 and at an opposing second end to second substrate 240. Connecting member 250 is fabricated from a suitable shape memory material such as described above in reference to strut 228 and/or strut 248. As shown in FIGS. 11 and 12, connecting member 250 includes a helical wire that biases first substrate 220 towards second substrate 240. Alternatively, connecting member 250 may include a U-shaped wire or a looped-shaped wire.

Referring to FIGS. 11 and 12, implantable medical device 210 is deployed at a target tissue site using a suitable catheter system. With first substrate 220 and second substrate 240 in the collapsed configuration, as shown in FIG. 12, implantable medical device 210 is delivered to the target tissue site. With implantable medical device 210 positioned at the target tissue site, such as positioned within a hole defined through a septum wall, first substrate 220 is released within a first chamber of the heart, i.e., the left atrium. Within the left atrium, first substrate 220 is deployed and moves from the collapsed configuration, as shown in FIG. 12, to the deployed configuration, as shown in FIG. 11. Second substrate 240 is then released within a second chamber of the heart, i.e., the right atrium. Within the right atrium, second substrate 240 is deployed and moves from the collapsed configuration to the deployed configuration. Connecting member 250 urges second substrate 240 towards first substrate 220 to retain implantable medical device 210 properly positioned within the septum wall with first substrate 220 properly positioned with respect to a surface of the septum wall within the left atrium and second substrate 240 properly positioned with respect to a surface of the septum wall within the right atrium. In this embodiment, an outer surface of sensor 224 and an outer surface of sensor 244 lay generally parallel to the respective heart chamber wall. In a particular embodiment, sensor 224 and sensor 244 are position about 0.5 mm to about 3.0 mm or, more specifically, about 1.0 mm to about 2.0 mm, from the respective heart chamber wall to prevent or limit cellular growth over the working components of sensor 224 and sensor 244, which may degrade or weaken signals transmitted from sensor 224 and sensor 244 to an external receiver (not shown).

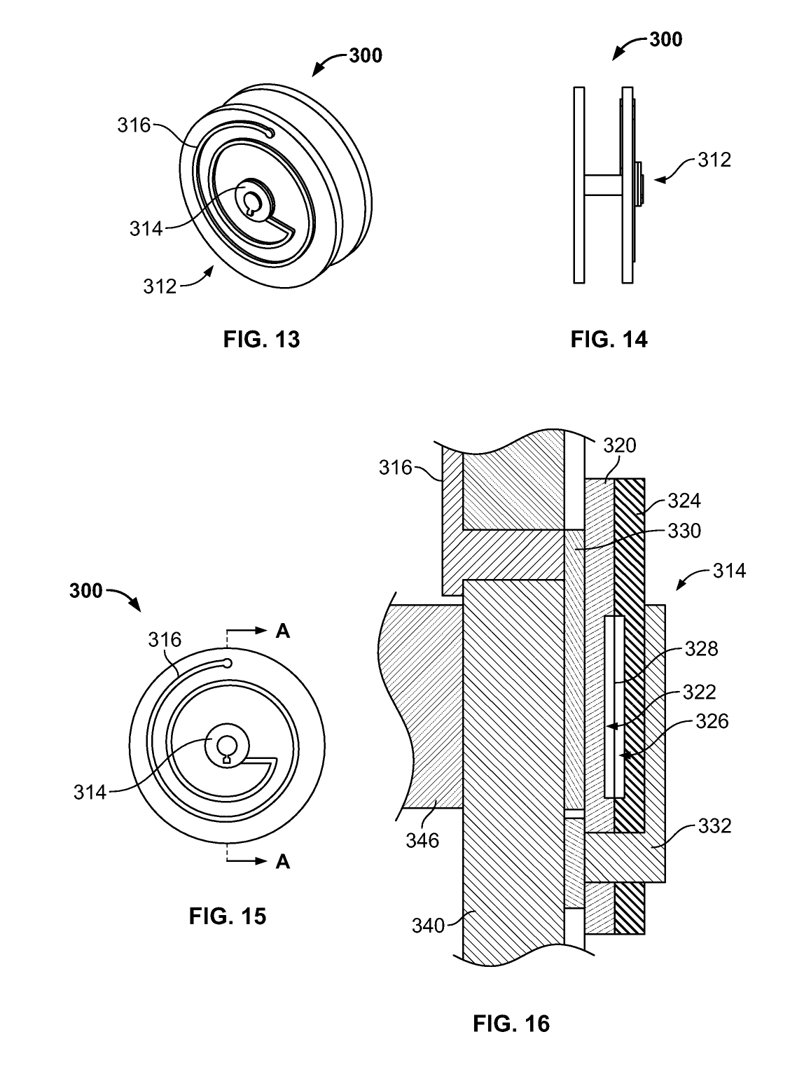

Referring to FIGS. 13-17, in one embodiment, an implantable medical device 300 includes a sensing unit 312 having a MEMS sensor 314 that is inductively coupled or electrically connected to a separate antenna 316. Sensor 314 includes a hermetically sealed chamber, an ASIC with a hermetically sealed chamber, or a hermetically sealed chamber electrically connected or inductively coupled to an ASIC that does not include an integral sensor. Sensor 314 and antenna 316 may be positioned on one or both sides of a septum wall. Further, sensor 314 and antenna 316 may be on the same surface or on opposing surfaces of the septum wall. One or more sensor 314 and one or more antenna 316 may optionally be on both surfaces of the septum wall. For example, in one embodiment, antenna 316 includes a suitable wire, such as a helical-configured wire, coupled to a single substrate that resides within and/or extends across the septum wall into one or both heart chambers. In a particular embodiment, the wire is fabricated of a suitable shape memory material such as described above. One or more sensors are operatively coupled to antenna 316 and sense a pressure, for example, within one or both heart chambers.

Sensor 314 may optionally be placed within the septum wall, wherein sensor 314 is rigid and has an outer diameter preferably less than approximately 2 mm. Sensor 314 positioned within the septum wall may be exposed to one or both adjoined chambers of the heart. Alternatively, sensor 314 may reside in the septum wall but be operatively coupled to one or both adjoined chambers of the heart. When sensor 314 is positioned in the septum wall, an anchoring mechanism is positioned on one or both sides of the septum wall such that the anchoring mechanism does not pass through the septum wall. Sensor 314 and antenna 316 may optionally be electrically connected or inductively coupled. Sensor 314 and/or antenna 316 may optionally be electrically connected or inductively coupled to an ASIC, wherein the ASIC may or may not include integrally formed sensors. In one embodiment, sensor 314 is a capacitive pressure sensor defining a hermetically sealed chamber fabricated with MEMS technology. The MEMS sensor does not contain signal processing electronics. In one embodiment, the MEMS sensor is electrically connected or inductively coupled to an ASIC that does not include an integral pressure sensor, and may optionally contain signal processing electronics to process the signal received from the MEMS sensor. In this embodiment, the ASIC may be electrically connected or inductively coupled to antenna 316 with sensor 314 electrically connected or inductively coupled to the ASIC. The MEMS sensor can be attached directly to the ASIC for sensing in the same location as the ASIC. The fabrication of MEMS sensors separately from a signal processing ASIC simplifies fabrication and avoids many of the challenges integrating MEMS with CMOS during fabrication.

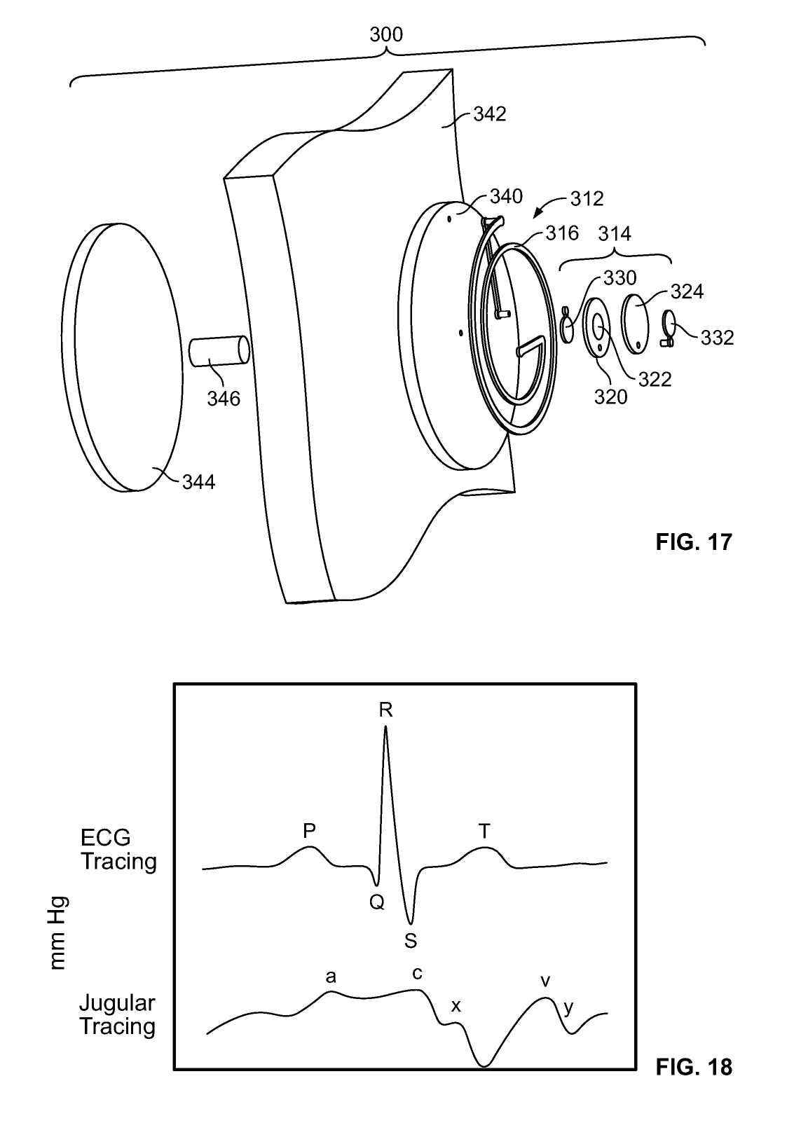

Referring further to FIGS. 16 and 17, in one embodiment, sensor 314 is inductively coupled or electrically connected to separate antenna 316. Sensor 314 includes a first portion 320 defining a void 322 and a second portion 324 defining a void 326 that is aligned with void 322 with first portion 320 coupled to second portion 324 to define a hermetically sealed chamber 328, as shown in FIG. 16. This description of sensor 314 with hermetically sealed chamber 328 is exemplary. It is within the scope of this invention that sensor 314 with hermetically sealed chamber 328 may be formed using any suitable process known to those skilled in the art and guided by the teachings herein provided. In one embodiment, first portion 320 and second portion 324 are formed of a suitable glass, silicon, or fused silica material. A first or bottom electrode 330 is positioned with respect to an outer surface of first portion 320 and operatively coupled to a first end of a patterned trace of conductor, such as including copper, forming antenna 316. A second or top electrode 332 is positioned with respect to an outer surface of second portion 324 and operatively coupled to a second end of the patterned trace of conductor, such as including copper, forming antenna 316. Bottom electrode 330 and top electrode 332 are formed of a suitable material, such as a copper or gold material. In one embodiment, antenna 316 is coupled to a first substrate 340. First substrate 340 is positioned with respect to a first surface of septum wall 342. A second substrate 344 is positioned with respect to an opposing second surface of septum wall 342 and coupled to first substrate 340 using a suitable connector 346. In one embodiment, first substrate 340, second substrate 344 and connector 346 are formed of a suitable material, such as a poly(tetrafluoroethylene) (PTFE) material.

In one embodiment, a reader device wirelessly resonates the sensor device. The resonating sensor device is energized by the reader using a periodic pulse of energy or, alternately, a periodic burst of energy at a frequency at or near the resonant frequency of the resonating sensor device. After energizing the sensor device, the reader amplifies the signal received from the sensor device through a tuned amplifier. The output of the tuned amplifier feeds a phase-locked-loop (PLL) circuit that includes a sample-and-hold (S/H) feedback amplifier circuit. The PLL circuit locks to the frequency of the received signal with the S/H circuit in sample mode. Prior to the received signal level dropping below the sensitivity threshold of the PLL, the S/H circuit is placed in hold mode. The S/H circuit remains in hold mode until the next signal reception from the sensor. As such, the S/H is used to update the PLL to the most recently received sensor frequency after each time the resonating sensor device is energized.

A counter circuit determines the frequency of the voltage controlled oscillator (VCO) that is used within the PLL for tracking the resonant frequency of the resonant sensor device. The counter circuit is synchronized to count frequency starting after the PLL has locked to the frequency of the received signal. Due to the fact that the accuracy of the counted frequency is higher than can be counted during the short period that the resonating sensor device is emitting a signal of sufficient amplitude for the reader to the signal's frequency, the S/H capability of the PLL causes the VCO frequency to remain fixed within the required accuracy level for the period needed for counting.

The counted frequency is provided for further processing by the reader device to determine the sensed parameter of the resonating sensor device. The sensed parameter may be then displayed, used in calculations, or used as part of a control algorithm. In one embodiment, a circuit is provided to dampen any resonance remnants in the reader antenna immediately after providing the burst of energy, then restoring some same or different quality factor (Q) to the reader antenna for reception of the resonating sensor device signal. Alternatively or in addition, a circuit is provided to modify the reader antenna between transmission and reception modes of operation, for example, positioning the reader antenna in a series resonant mode during transmission and in a parallel resonant node during reception.

In one embodiment, a reader tuned amplifier using strictly resistor-capacitor (RC) high-pass and low-pass circuitry, as opposed to inductor-capacitor (LC) circuitry, and no feedback circuitry is provided. This circuit design is chosen to enhance the transient response of the reader tuned amplifier while providing significant frequency discrimination in the tuned amplifier output. A direct amplification of the received signal is preferred in the current reader due to frequency and amplifier considerations, although a radio frequency mixer and a sum-frequency or difference-frequency tuned amplifier might be used alternatively in other applications.

A PLL circuit is operatively coupled to the tuned amplifier and employs a frequency divider for the frequency input from the tuned amplifier to allow, for example, a particular VCO choice for the PLL. Further, the PLL circuit may employ a frequency divider for the VCO frequency output to facilitate the use of a higher VCO frequency than the resonating sensor device resonant frequency. A higher VCO frequency than reader received resonating sensor frequency allows faster frequency counting to full resolution at a given resonating sensor device frequency. Alternately, this divider in addition to the tuned amplifier input divider allows the choice of a VCO at any desired center frequency for operation.

In a particular embodiment, the reader circuitry includes at least one ASIC to implement portions of the reader circuitry. In particular, the reader may include a sequencing timer to sequentially control pulse transmission, damp antenna resonance, configure the reader antenna, enable the reader tuned amplifier, control sample and hold timing on the S/H circuit, initiate counting of the VCO frequency, complete counting of the VCO frequency including storage of results to a data buffer, and report the frequency count complete. This circuit might also include sub-circuits to assist in configuring the VCO, adjust timing as needed to optimize reader circuit operation, initiate the sample sequence on a periodic basis, provide a complete processor based system to control the reader operation, provide display of sensed data, and transfer of sensed data to external systems.

The reader circuitry may also include at least one programmable array circuit to implement portions of the reader circuitry. In particular, the reader may include a sequence timer to sequentially control pulse transmission, damp antenna resonance, configure the reader antenna, enable the reader tuned amplifier, control sample and hold timing on the S/H circuit, initiate counting of the VCO frequency, complete counting of the VCO frequency including storage of results to a data buffer, and report the frequency count complete. This circuit might also include sub-circuits to assist in configuring the VCO, adjust timing as needed to optimize reader circuit operation, initiate the sample sequence on a periodic basis, provide a complete processor based system to control the reader operation, provide display of sensed data, and transfer of sensed data to external systems.

Further, the reader tuned amplifier may employ signal clamping circuitry to limit stage voltages preventing stage saturation in the band pass filters to ensure effective filtering of signal, and significantly improvement the dynamic range of the tuned amplifier.

In one embodiment, a backing plate includes conducting protrusions, such as metallic protrusions, to facilitate electrical contact between the electrical apparatus of the sensor and the patient's heart. The electrical contact allows the electrical current within the myocardium to be directed into the electrical system of the sensor to power the system and/or charge the capacitor of the LC tank. Further, the metallic protrusions facilitate sensing an electrical rhythm and correlating the sensed rhythm to a sensed pressure to provide feedback such that a contractile rate of a chamber being sensed can be utilized in a therapeutic manner. One of the metallic protrusions may be a ground completing the electrical circuit. The electrical rhythm transmitted includes a combination of polarization and depolarization of the myocardium generated by the influx and efflux of cations and anions within the myocardium. The charge differential and subsequent electrical current may be transmitted as an integrated data package along with the pressure. Additionally, the backing plate metallic protrusions may be coated in a drug eluting compound including, without limitation, a steroid or a slow release drug polymer that inhibit scar formation or cellular hyperplasia.

In one embodiment, multiple sensors positioned within different chambers are timed based on electrical signals and pressures sensed to assist in the coordinated flow of blood through the various chambers of the heart, thereby creating a normal or near normal physiologic status. The electrical signal from a sensor placed in a vicinity of the sinoatrial node sensing a local depolarization can be timed with a similar sensor placed in a vicinity of an atrioventricular node bundle along the neural pathways of the heart. The coordinated electrical and pressure signals of the heart are optimized for ideal pressures and rhythm.

In one embodiment, an electrical and pressure sensing system inhibits abnormal rhythms sensed by a discharge of an electrical capacitive charge either as an electrical pulse, based on the charge stored, or in a continuous fashion as determined by a continuous external power source timed to discharge in synchronous or dissynchronyous fashion.

In one embodiment, the sensors are placed on an external surface of the heart to detect an intra-cavity pressure of a pericardial chamber. Sensors placed on the external surface of the heart include metallic protrusions that extend within the myocardium with the electrical current of the myocardium providing charge to and storage within the capacitor.

In one embodiment, the Right Atrial Pressure (RAP) and the Left Atrial Pressure (LAP) are monitored using a method including an algorithm developed to reflect changes in the ventricular function of a failing heart. The algorithm may be derived from and at least partially based on a healthy functioning heart. However, pressures within the heart and pressure relationships to the QRS pattern will vary from patient to patient and over time. FIG. 18 illustrates the RAP waveform signal for a healthy functioning heart.

Referring to FIG. 18, normal sinus rhythm can be described as including three positive waves, namely ‘a’, ‘c’, and ‘v’ waves and two negative waves, namely ‘x’ and ‘y’ descents. The ‘a’ wave represents an increased atrial pressure and correlates with the P wave on the corresponding ECG. The ‘c’ wave represents a slight elevation of the tricuspid valve into the right atrium during early ventricular contraction. The ‘v’ wave represents the filling of the right atrium against a closed tricuspid valve. The ‘x descent’ represents the downward movement of the ventricle during systolic contraction. The ‘y descent’ represents the opening of the tricuspid valve and the flow of blood into the right ventricle.

A LAP signal represents left atrial pressure during normal sinus rhythm and can be described by classical features of the LAP signal. The ‘v’ wave corresponds to the opening of the mitral valve. The ‘a’ wave corresponds to atrial contraction. The ‘c’ wave corresponds to isovolumic contraction and the sequential opening of the aortic valve. The corresponding left atrium volume decreases following the opening of the mitral valve and again at atrial contraction as the left atrium empties its contents into the left ventricle.

The ‘z’ point (not shown) represents a local minimum value following atrial contraction and closure of the mitral valve immediately preceding ventricular systole. The negative x-wave characteristic or ‘x descent’ (or systolic collapse) is associated with the descent of the mitral ring during ventricular ejection. The ‘y descent’ (or diastolic collapse) following the opening of the mitral valve is associated with early ventricular filling and passive atrial emptying.

The diagnosis of various heart conditions requires the monitoring of both peripheral blood pressure and intra-cardiac pressures. Conventional methods for monitoring intra-cardiac pressures are limited to the placement of catheters at the time of cardiac catheterization or the placement of a swan-ganz catheter. The swan-ganz catheter is placed on the right side of the heart through the central veins to directly monitor the right atrial pressure. The catheter tip is placed in the pulmonary artery and once the catheter tip is positioned within the pulmonary artery, the catheter tip indirectly measures the left ventricular pressure with the mitral valve open during ventricular filling.

The placement of a transseptal device that is able to directly monitor the RAP and the LAP simultaneously allows for the best surrogates for the right ventricular end diastolic pressure (RVEDP) and the left ventricular end diastolic pressure (LVEDP) to be detected or sensed for monitoring a failing heart.

By placing a device with respect to the pulmonary artery to monitor the pulmonary artery pressure, measurements of the pulmonary artery systolic pressure, PA (sys), and the pulmonary artery diastolic pressure, PA (dia), are obtained. PA (sys) is a composite of the right ventricular systolic pressure and the resistance seen across the pulmonary vascular bed. PA (dia) is seen as a surrogate for left ventricular end diastolic pressure when the mitral valve is open, as represented by the ‘v’ wave in FIG. 18.

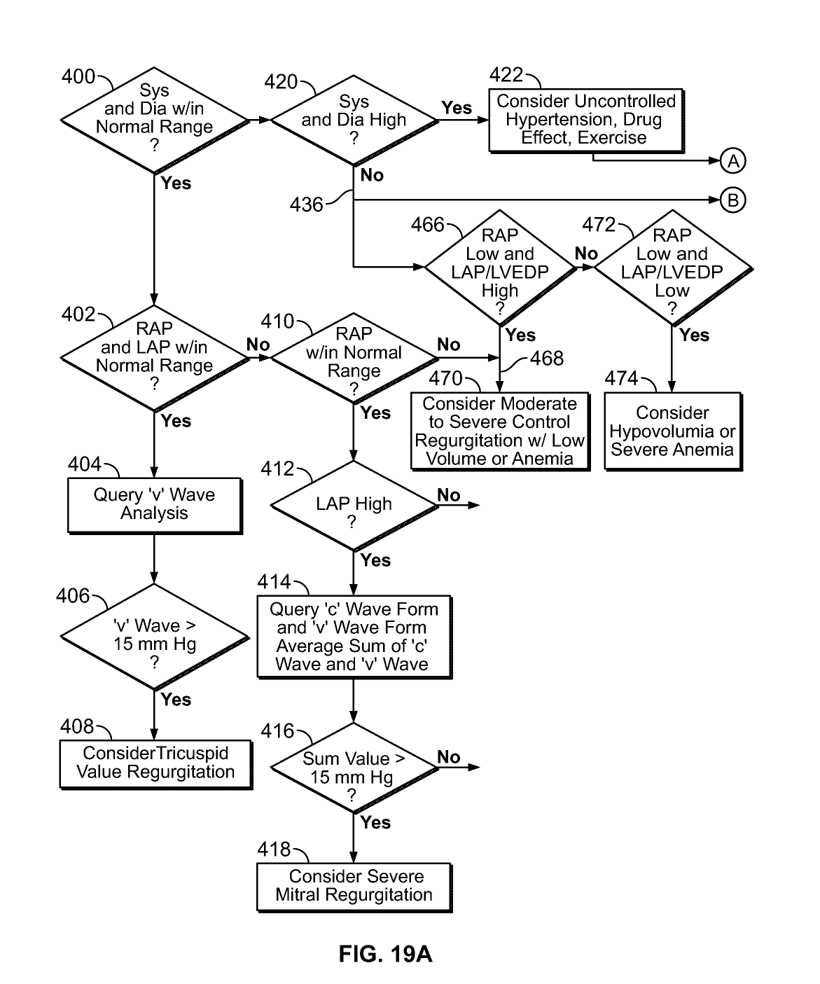

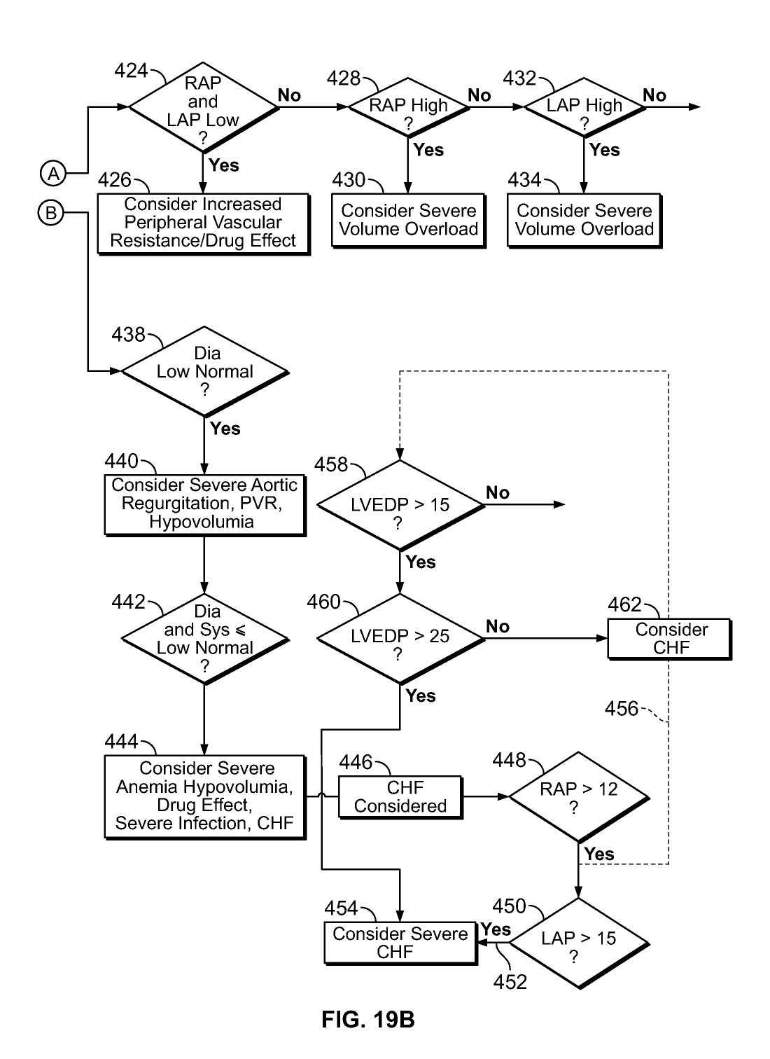

FIGS. 19A and 19B show a flow diagram describing steps of a method to facilitate a diagnosis of a heart condition. In one embodiment an algorithm facilitates diagnosis of a heart condition. The following definitions and constraints are utilized in the described algorithm.

### Definitions and Constraints

### Males:

Systolic blood pressure (sys) normal range 90<sys<140 mmHg
Diastolic blood pressure (dia) normal range 50<dia<90 mmHg

### Females:

Systolic blood pressure (sys) normal range 70<sys<140 mmHg
Diastolic blood pressure (dia) normal range 40<dia<90 mmHg
RAP range 0 mmHg to 12 mmHg
LAP range 0 mmHg to 12 mmHg
RAP high value greater than 12 mmHg
LAP high value greater than 12 mmHg

### RAP:

‘a’ wave values 2-4 mmHg
‘c’ wave values 4-6 mmHg
‘v’ wave values 6-10 mmHg

### LAP:

‘a’ wave values 2-4 mmHg
‘c’ wave values 4-6 mmHg
‘v’ wave values 6-10 mmHg

### LVEDP:

normal range 0-12 mmHg
Moderately High>12 mmHg
Severe>15 mmHg

A controller (not shown) includes a processor, a memory, a plurality of input channels, and a plurality of output channels and a computer. As used herein, the term computer is not limited to just those integrated circuits referred to in the art as a computer, but broadly refers to a processor, a microcontroller, a microcomputer, a programmable logic controller, an application specific integrated circuit, and other programmable circuits, and these terms are used interchangeably herein. In the exemplary embodiment, memory may include, without limitation, a computer-readable medium, such as a random access memory. Alternatively, a floppy disk, a compact disc, read only memory (CD-ROM), a magneto-optical disk (MOD), and/or a digital versatile disc (DVD) may also be used. Also, in the exemplary embodiment, a plurality of input channels may represent, without limitation, computer peripherals associated with an operator interface such as a mouse and a keyboard. Alternatively, other computer peripherals may also be used, for example, a scanner. Further, in the exemplary embodiment, a plurality of output channels may include, without limitation, an operator interface monitor.

The controller receives a plurality of inputs from a plurality of sensors, processes the inputs, generates appropriate outputs based on programmed algorithms and discrete circumstances, and transmits signals to the appropriate system components. In one embodiment, the controller receives data from a user, and is configured to run the following algorithm and display the data.

If the systolic pressure and the diastolic pressure are within a normal range 400, 90<sys<140 mmHg for a male patient, for example, and the RAP and the LAP are within a normal range 402, then query ‘v’ wave analysis 404. If the ‘v’ wave is determined to be greater then 15 mmHg 406, tricuspid valve regurgitation is considered 408. If the RAP is within a normal range 410 and the LAP is high 162, query the ‘c’ wave form and the ‘v’ wave form and average the sum of the ‘c’ wave and the ‘v’ wave 414. If the sum value of the ‘c’ wave and the ‘v’ wave is greater than 15 mmHg 416, query severe mitral regurgitation 418.

If the systolic pressure and the diastolic pressure are high 420, that is, greater than or equal to a high normal, as calculated by averaging every 60^{th} systolic peak value and every 60^{th} diastolic trough value for 24 hours, then consider uncontrolled hypertension, drug effect, and exercise 422. Additionally, if the RAP is low and the LAP is low 424, then consider increased peripheral vascular resistance or drug effect 426.

If the RAP is high 428, then consider severe volume overload 430. If the RAP is not high but the LAP is high 432, then consider severe volume overload 434.

In one embodiment, if the systolic pressure and the diastolic pressure are not high 436, that is, less than a high normal, consider whether the diastolic pressure is low normal 438. If the diastolic pressure is low normal, then consider severe aortic regurgitation, high peripheral vascular resistance (PVR) and hypovolumia 440.

If systolic pressure and diastolic pressure are low 442, that is, less than or equal to the low normal, as calculated by the averaging of every 60^{th} systolic peak value and every 60^{th} diastolic trough value for 24 hours, then consider severe anemia, hypovolumia, drug effect, severe infection, such as sepsis and CHF 444.

If CHF is considered 446, then query for the RAP and the LAP. If a value of the RAP above 12 is obtained 448, then query the LAP 450. If a value of the LAP above 15 is obtained 452, then consider severe CHF 454.

Alternatively, if a value of the RAP above 12 is obtained and the LVEDP is obtained 456 instead of the LAP, determine 458 whether the LVEDP value is greater than 15. If the LVEDP value is greater than 15 but not greater than 25 (step 460), then consider moderate CHF 462. If the LVEDP value is greater than 25 (step 464), then consider severe CHF 454.

If the systolic pressure and the diastolic pressure are low, consider whether the RAP is low and the LAP or the LVEDF is high 466. If the RAP is low and the LAP or the LVEDP is high 468, then consider moderate to severe mitral regurgitation with low volume or anemia 470. If the systolic pressure and the diastolic pressure are low and the RAP is low and the LAP or the LVEDP is low 472, then consider hypovolumia or severe anemia 474.

In one embodiment, the signals are averaged across a 24 hour cycle, a 7 day cycle and/or a 30 day cycle. The rate of rise of the values constitutes a DP/DT (change in pressure divided by change in time) value that reflects a change in the function of the heart. If the DP/DT for the RAP or the LAP is greater than 5 mmHg in a 24 hour cycle, then consider early CHF. If the DP/DT for the RAP or the LAP is greater than 10 mmHg in a 7 day cycle, then consider early CHF. If the DP/DT for the RAP or the LAP is greater than 15 mmHg in a 30 day cycle, then consider early CHF.

Based of the analysis of the timing of the troughs for the ‘x’ descent and the ‘y’ descent, upon detection of the waveforms, an analysis is made to determine a rate at which the ‘x’ descent and the ‘y’ descent occur and a calculation can be made to infer the atrial rate. Movement of the atrial septum has to be accounted for and the pressure waveform generated by a movement of the sensor across the atrial cavity within the blood. The rhythmic movement of the atrial septum may cause an overall rhythmic fluctuation of the RAP and the LAP waveforms in the context of the individual ‘a’ wave, ‘c’ wave and ‘v’ wave.

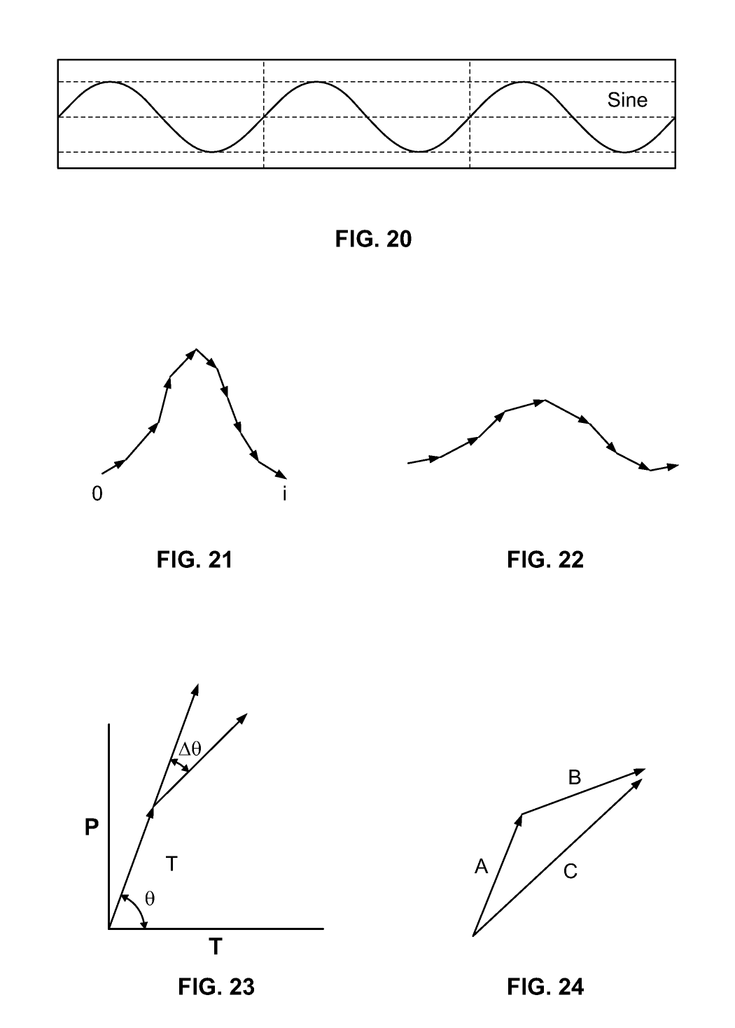

In one embodiment, a respiratory variation is superimposed on the waveforms and calibrated into the analysis of the atrial waveforms. If the atrial fibrillation rhythm is present, the rhythm onset is detected in a change in the timing of the inflexion points ‘x1’ to ‘x2’ and ‘y1’ to ‘y2’. Once detected, the measurement of the direct values of atrial pressure is determined by averaging a median point of the wave ascents as the wave ascents are sensed. An average of the sensed wave ascents over one minute intervals sampled every 60 minutes for 4 hours are utilized to calculate an average RAP pressure and an average LAP pressure. Most blood pressure waveforms are described in a sinusoidal pattern, as shown in FIG. 20, which all conform to the basic equation:

y(t)=A·sin(ωt+θ) (1)

This describes a sine wavelike function of time (t), wherein A=a peak deviation from center, ω=angular frequency (radians/sec.), and θ=a phase of an angle θ.

Thus, assuming a finite number of points which by summation constitutes the sinusoidal waveform described by the equation (1), a change in vectors between each point governs an overall shape of the curve, as shown in FIGS. 21 and 22. A change in a vector between each point is governed by the instantaneous effect of a summation of an infinite number of internal and external variables on the direction of the vector to give the final blood pressure waveform. Each vector point is added to the next to make up the blood pressure wave form. Thus, under the assumption that a summation of forces on a final vector (direction) of the blood pressure curve is related to the eventual angle of the vector as shown in FIGS. 23 and 24, a tangent of θ is described by the equation (2):

tan θ=ratio of P/T, (2)

Assuming that the summation of the two vectors, A and B, as shown in FIG. 24, A+B=C, the sum of the vectors assumes that their angles are also additive and, thus, a change in θ ratio approximates to dP/dT.

Assuming that dP/dT approximates tan θ=tan(θ+2πK), the sum of external and internal forces (position, activity state, emotional). The final vector of the blood pressure curve between (0−i) will be represented by:

$${\sum }_{□ = □}^{□} \text{?} \{ Δ ( d p / d t ) \} = {\sum }_{□ = □}^{□} \text{?} tan Δ θ = {\sum }_{□ = □}^{□} \text{?} tan ( Δ θ + 2 π K ) \\ \text{?} \text{indicates text missing or illegible when filed} \qquad ( 3 )$$

The external and internal forces affecting a change in the vector angle can then be accounted for. This change, Δ0, in vector angle is based on a change in a resistance to flow which is governed by two forces, namely, (1) a wall stress of the left ventricle that is equal to a peak systolic stress and an integral of left ventricular wall stress over time, and ac, (limiting force to left ventricular shortening; and (2) systemic vascular resistance (SVR), that is equal to 80 (MAP-RAP)/Cardiac Output (CO).

Wall Stress of the left ventricle is governed by a multitude of constant varying factors, such as a presence of coronary artery disease, valvular disease or intrinsic myocyte deficiencies. The wall stress is best estimated on ECHO by the global ejection fraction or by surrogates, such as left ventricular end-diastolic pressure or shortening time. If LVEDP is used, then a left atrial placed pressure monitoring device will allow for easy measurement at the end of diastole when the Mitral Valve is open. The following basic assumptions are made:

1. σₑₛ cannot be measured in real time;
2. most of the parameters for SVR can be measured and calculated;
3. Total Resistance is equal to σₑₛ+SVR (Wall stress left ventricle+Systemic Vascular Resistance)
Based on the assumptions the following equation is derived:

$$S V R = \frac{D P + \frac{1}{3} ( P P )}{H R \times A U C} \qquad ( 4 )$$

wherein DP=Diastolic blood pressure (measured);
PP=Pulse Pressure (calculated from measured systolic blood pressure and diastolic blood pressure);
HR=Heart rate (measured from left ventricular rate assuming normal sinus rhythm this will be the actual heart rate);
AUC=Area under the curve (the integral of the parabolic function of the blood pressure curve); and
Θ=the angle between each point on the blood pressure curve assuming 300×600 point resolution.

As such, the SVR affects a direction of vectors that make up a direction of the vectors,

SVR(tan θ) (5)

this then becomes:

$$\frac{D P + \frac{1}{3} ( P P )}{H R \times A U C} ( {\sum }_{0 - i}^{n} tan ( Δ θ + 2 π K ) ) \qquad ( 6 )$$

A linear mixed model analyzing the extracted data with correction for baseline variability can allow for determination of the changes in time (longitudinal effects) of blood pressure.

In one embodiment, a system for diagnosing a heart condition includes a transseptal device positioned with respect to a pulmonary artery to monitor at least one flow characteristic of blood through the pulmonary artery and operatively coupled to an external transceiver or reader. The transseptal device is configured to generate at least one signal representative of the at least one flow characteristic to facilitate monitoring a right atrial pressure (RAP) and a left atrial pressure (LAP). The external reader is configured to receive the at least one generated signal from the transseptal device representative of the at least one flow characteristic; process the at least one received signals; and generate an appropriate output representative of the at least one flow characteristic of blood through the pulmonary artery.

In one embodiment, the transseptal device includes at least one sensor configured to sense one of at least one of a physical, chemical and physiological parameter within a respective heart chamber to facilitate obtaining data for at least one of cardiac blood pressure analysis, temperature analysis, blood chemical analysis, blood osmolar analysis, and cellular count analysis. The transseptal device includes a plurality of sensors each configured to transmit measurement data wirelessly to the external reader and the external reader includes an RF filter operatively coupled to the plurality of sensors. The external reader may include a processor, a memory, a plurality of input channels, a plurality of output channels and/or a computer. The external reader receives data from a user, and is further configured to run an algorithm to facilitate diagnosing a heart condition and display data.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for monitoring a right atrial pressure (RAP) and a left atrial pressure (LAP) for diagnosis of a heart condition, the method comprising:
positioning a transseptal device with respect to a pulmonary artery to monitor at least one flow characteristic of blood through the pulmonary artery, the transseptal device configured to generate one or more signals representative of the at least one flow characteristic; and
detecting a right ventricular end diastolic pressure (RVEDP) and a left ventricular end diastolic pressure (LVEDP) to facilitate monitoring the heart condition.

2. A method in accordance with claim 1 wherein positioning a transseptal device with respect to a pulmonary artery facilitates monitoring at least one of a pulmonary artery pressure, a measurement of a pulmonary artery systolic pressure, and pulmonary artery diastolic pressure.

3. A method in accordance with claim 1 wherein positioning a transseptal device with respect to a pulmonary artery to monitor at least one flow characteristic of blood through the pulmonary artery monitoring a pulmonary artery systolic pressure as a composite of a right ventricular systolic pressure and a resistance across a pulmonary vascular bed.

4. A method in accordance with claim 1 wherein the pulmonary artery diastolic pressure is representative of a left ventricular end diastolic pressure when a mitral valve is open.

5. A method in accordance with claim 1 further comprising determining whether a systolic pressure and a diastolic pressure are within a normal range and the RAP and the LAP are within a normal range.

6. A method in accordance with claim 5 further comprising querying a ‘v’ wave analysis if the systolic pressure and the diastolic pressure are within a normal range and the RAP and the LAP are within a normal range.

7. A method in accordance with claim 5 further comprising considering tricuspid valve regurgitation if the ‘v’ wave is determined to be greater than 15 mmHg.

8. A method in accordance with claim 5 wherein, if the RAP is within a normal range and the LAP is high, the method further comprising querying a ‘c’ wave form and a ‘v’ wave form and averaging a sum of a ‘c’ wave and a ‘v’ wave.

9. A method in accordance with claim 8 further comprising querying a severe mitral regurgitation if the sum value of the ‘c’ wave and the ‘v’ wave is greater than 15 mmHg.

10. A method in accordance with claim 5 further comprising determining whether the systolic pressure and the diastolic pressure is greater than or equal to a high normal, as calculated by averaging every 60^{th} systolic peak value and every 60^{th} diastolic trough value for 24 hours.

11. A method in accordance with claim 10 wherein, if the systolic pressure and the diastolic pressure is greater than or equal to a high normal, the method further comprising considering uncontrolled hypertension, drug effect, and exercise.

12. A method in accordance with claim 11 wherein, if and the RAP is low and the LAP is low, the method further comprising considering an increased peripheral vascular resistance and a drug effect.

13. A method in accordance with claim 11 wherein, if one of the RAP and the LAP is high, the method further comprising considering severe volume overload.

14. A method in accordance with claim 10 wherein, if the systolic pressure and the diastolic pressure is less than a high normal and the diastolic pressure is a low normal, the method further comprising considering severe aortic regurgitation, high peripheral vascular resistance and hypovolumia.

15. A method in accordance with claim 14 wherein, if the systolic pressure and diastolic pressure are less than or equal to a low normal, as calculated by an averaging of every 60^{th} systolic peak value and every 60^{th} diastolic trough value for 24 hours, the method further comprising considering severe anemia, hypovolumia, drug effect, severe infection, and chronic heart failure (CHF).

16. A method in accordance with claim 15 wherein, if CHF is considered and a value of the RAP is above 12, the method further comprising querying the LAP and, if a value of the LAP is above 15, considering severe CHF.

17. A method in accordance with claim 15 wherein, if CHF is considered and a value of the RAP is above 12, the method comprising obtaining the LVEDP instead of the LAP and determining whether the LVEDP value is greater than 15.

18. A method in accordance with claim 17 wherein, if the LVEDP value is greater than 15 but not greater than 25, the method further comprising considering moderate CHF and if the LVEDP value is greater than 25, then considering severe CHF.

19. A method in accordance with claim 10 wherein, if the systolic pressure and the diastolic pressure is less than a high normal, the method further comprising considering whether the RAP is low and at least one of the LAP and the LVEDF are high.

20. A method in accordance with claim 19 wherein, if the RAP is low and at least one of the LAP and the LVEDP is high, the method further comprising considering moderate to severe mitral regurgitation with one of low volume and anemia.

21. A method in accordance with claim 19 wherein, if the RAP is low and at least one of the LAP and the LVEDP is low, the method further comprising considering hypovolumia and severe anemia.

22. A method in accordance with claim 1 further comprising averaging the one or more signals generated by the transseptal device and representative of the at least one flow characteristic across a time cycle, wherein a rate of rise of a value is representative of a change in pressure divided by a change in time value that reflects a change in a function of the heart.

23. A method in accordance with claim 22 wherein, if a rate of rise for at least one of the RAP and the LAP is greater than 5 mmHg in a 24 hour cycle, the method further comprising considering an early CHF.

24. A method in accordance with claim 22 wherein, if a rate of rise for at least one of the RAP and the LAP is greater than 10 mmHg in a 7 day cycle, the method further comprising considering an early CHF.

25. A method in accordance with claim 22 wherein, if a rate of rise for at least one of the RAP and the LAP is greater than 15 mmHg in a 30 day cycle, the method further comprising considering an early CHF.

26. A method in accordance with claim 22 further comprising inferring an atrial rate

27. A system for diagnosing a heart condition, the system comprising:
a transseptal device positioned with respect to a pulmonary artery to monitor at least one flow characteristic of blood through the pulmonary artery, the transseptal device configured to generate at least one signal representative of the at least one flow characteristic to facilitate monitoring a right atrial pressure (RAP) and a left atrial pressure (LAP); and
an external reader operatively coupled to the transseptal device, the external reader configured to:
receive the at least one generated signal from the transseptal device representative of the at least one flow characteristic;
process the at least one received signals; and
generate an appropriate output representative of the at least one flow characteristic of blood through the pulmonary artery.

28. A system in accordance with claim 27 wherein the transseptal device comprises at least one sensor configured to sense one of at least one of a physical, chemical and physiological parameter within a respective heart chamber to facilitate obtaining data for at least one of cardiac blood pressure analysis, temperature analysis, blood chemical analysis, blood osmolar analysis, and cellular count analysis.

29. A system in accordance with claim 28 wherein the transseptal device is configured to transmit measurement data wirelessly to the external reader.

30. A system in accordance with claim 28 wherein the external reader comprises an RF filter operatively coupled to a plurality of sensors.

31. A system in accordance with claim 27 wherein the external reader comprises a processor, a memory, a plurality of input channels, and a plurality of output channels and a computer.

32. A system in accordance with claim 27 wherein the external reader receives data from a user, and is further configured to run an algorithm to facilitate diagnosing a heart condition and display data.
